(19) United States
(12) Patent Application Publication
(10) Pub. No.: US 2010/0119468 A1
(43) Pub. Date: May 13, 2010

(54) CATIONIC CONDITIONING AGENT

(75) Inventors: Ivette Garcia Castro, Ludwigshafen (DE); Hubertus Peter Bell, Mannheim (DE); Olga Pinneker, Hessheim (DE); Claudia Wood, Weinheim (DE); Gabi Winter, Wachenheim (DE); Nicole Meier, Mannheim (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/524,924

(22) PCT Filed: Jan. 31, 2008
(86) PCT No.: PCT/EP2008/051221

§ 371 Date: Jul. 29, 2009

(30) Foreign Application Priority Data
Jan. 31, 2007 (EP) 07101486.4

(51) Int. Cl. A61K 8/72; A61Q 5/00; A61Q 5/02; A61Q 19/00

(52) U.S. Cl. 424/70.11; 424/78.03

(57) ABSTRACT

The present invention relates to compositions comprising at least one polymer a) having cationic and/or cationogenic groups and a molecular weight M_{w} in the range from 10 000 to 5 million and at least one polymer b) which has been prepared in the presence of polymer a).

The present invention relates to compositions comprising at least one polymer a) having cationic and/or cationogenic groups and a molecular weight M_{w} in the range from 10 000 to 5 million and at least one polymer b) which has been prepared in the presence of polymer a).

Furthermore, the present invention relates to skin and hair cosmetic compositions, in particular shampoos and other haircare compositions, comprising the polymers a) and b), where b) has been prepared in the presence of a). Accordingly, the invention relates to compositions for the cleansing and/or care of the hair. In particular, apart from shampoos, the invention also relates to further haircare compositions which are selected from the group consisting of pretreatment compositions, hair rinses, hair conditioners, hair balms, leave-on hair treatments, rinse-off hair treatments, hair tonics, pomades, styling creams, styling lotions, styling gels, end fluids, hot-oil treatments and foam treatments.

### PRIOR ART

Haircare compositions serve primarily to improve the dry and wet combability, the feel to the touch, the shine and the appearance of the hair, and also to impart antistatic properties to the hair.

A shampoo should lather and cleanse the hair well, be mild and compatible and also practicable and pleasant to handle, it must also contribute to the care of the hair or to the elimination of hair and scalp problems. These additional effects and the cleansing effect, which has become self-evident, are characteristic of a modern shampoo. Cleansing power and foamability, skin compatibility, ability to be thickened and hydrolysis stability of the individual ingredients of shampoos are heavily dependent on the pH. The ingredients used in shampoos should optimally develop these properties in the neutral and weakly acidic pH range (pH 5-7), but outside of this pH range, should also exhibit no significant losses in performance. The selected ingredients of the shampoos must be chemically stable and compatible with all of the other formulation constituents so that, for example, no reductions in effect or separations take place. Shampoos are expected to have an adequate cleansing power coupled with not too strong a degreasing effect and simultaneously adequate mildness. It is important that the cleansing power and the other desired surfactant properties are present both in soft water and in hard water. Shampoos have to be well tolerated by skin and mucosa and must therefore have no aggressive effect under the customary use conditions. A good cleansing performance does not have to be linked to considerable foam formation. Nevertheless, foam quantity and quality of the shampoos during washing represent important criteria for the consumer which have to be satisfied by the shampoos. Besides the cleansing effect, shampoos also have a conditioning effect which is ensured through the content of conditioners in the shampoo. Conditioners are auxiliaries which attach to the hair and remain on the hair even after the rinsing process. They lead to an improvement in combability, feel and shine of the hair. In the case of certain hair types (fine hair) or overdosing, however, the conditioners can also lead to undesired weighing down of the hair. From a formulation point of view, this means that when using conditioners, it is always necessary to ensure a good balance between conditioning performance and weighing-down of the hair. Furthermore, when using conditioners, it should also be ensured that the regular application of the product does not lead to a continuously growing amount of conditioners on the hair (build-up effect). The provision of products with a complex profile of properties often presents difficulties. Such complex property profiles often require the use of numerous ingredients in one preparation, which in turn implies the risk of incompatibility toward one or more of these ingredients on the part of the consumer.

Conditioners in shampoos are primarily silicones and cationic polymers.

Silicones have the disadvantage that they are mostly water-insoluble and the shampoo formulation has to be stabilized through dispersants. These additives are often undesired. In addition, silicones sometimes exhibit considerable build-up effects and, following repeated use, the hair feels unpleasantly weighed down.

Many cationic polymers which are used as conditioners in shampoos, such as, for example, cationic cellulose derivatives, form surfactant-polymer complexes with anionic surfactants in the shampoo formulation; these are water-insoluble if the charge density of the polymers is high. For this reason, use is usually made of cationic polymers with low charge density so that they are soluble in the formulation.

However, cationic polymers with a high charge density have a greater affinity to the hair, for which reason it is desirable to use highly charged polymers in shampoos. However, the surfactant-polymer complexes in the formulation are then insoluble. The formulation has to be stabilized through the addition of dispersion auxiliaries.

WO 94/06403 describes the use of, inter alia, copolymers of N-vinylpyrrolidione and 3-methyl-1-vinylimidazolium salts with high charge density in combination with further water-insoluble conditioners in shampoo formulations. Accordingly, dispersants are used for stabilizing the formulations.

WO 94/06409 and U.S. Pat. No. 5,580,494 describe shampoo compositions based on an alpha-olefinsulfonate as detergent and a cationic polymer with a high charge density, e.g. copolymers of N-vinylpyrroiidone and 3-methyl-1-vinylimidazolium salts as conditioners. Dispersion auxiliaries also have to be added here to stabilize the formulations.

EPA 246 580 describes that quaternized vinylimidazole copolymers with various other monomers are used as hair conditioners. The polymers described therein have the disadvantage that, in the case of a low fraction of the quaternized vinylimidazole monomers in the presence of anionic surfactants, they a low effect, and in the case of a high fraction of the quaternized vinylimidazole, no stable dispersions form.

EP-A 911 018 describes the use of cationic copolymers obtainable by free-radically initiated copolymerization of

(a) 60 to 99 mol %, preferably 65 to 95 mol % and, particularly preferably, 70 to 90 mol % of an optionally substituted 1-vinylimidazole or a quaternized 1-vinylimidazole,

(b) 1 to 40 mol %, preferably 5 to 35 mol %, particularly preferably 10 to 30 mol %, of an acid comprising a polymerizable double bond, or salts thereof and

(c) 0 to 30 mol %, preferably 0 to 20 mol %, particularly preferably 0 to 10 mol % of a further free-radically copolymerizable monomer

and subsequent quaternization of the polymer if a nonquaternized 1-vinylimidazole is used as monomer (a), as active ingredients in hair cosmetic preparations, in particular as conditioners in shampoos.

WO 02/083073 describes water-soluble polymer complexes where, in the polymerization, a water-soluble host polymer is initially introduced, and one or more water-soluble monomers are polymerized so that a stable single-phase aqueous system is formed. Compositions according to claim 1 are not described.

The compositions which are known from the prior art have neither satisfactory haircare properties, such as, for example, good wet combability, nor satisfactory formulation-related properties, such as, for example, good stability.

In the case of numerous cationic polymers from the prior art, incompatibilities arise with typical charge-balancing stoichiometric quantitative ratios of polymer to surfactant.

These are evident, for example, from diminishing transparency, i.e. from clouding of the composition which arises.

It was an object of the present invention to provide aqueous compositions in the form of a shampoo or skin cleansing composition which have a very good conditioning effect for hair, skin and nails and at the same time have long-term stability in the presence of anionic surfactants.

There is therefore still a need for shampoos and haircare compositions which at the same time impart to the hair good sensorily detectable properties, such as elasticity, a pleasant feel and volume, without a good conditioning and cleansing effect being accompanied by an unsatisfactory greasy and/or sticky appearance of the hair treated therewith and which are in the form of formulations with long-term stability and, furthermore, good foaming ability and foam quality.

The aim was to develop shampoos and haircare compositions with the abovementioned properties on the basis of the fewest possible feed materials since, in the case of compositions from the prior art, the large number of necessary components sometimes leads to skin irritations, allergic reactions and other incompatibilities. Particularly in the field of children and baby shampoos and haircare compositions, there is also a requirement for compositions with the smallest possible number of different ingredients.

It was an object of the present invention to provide compositions which produce, without additional dispersion auxiliaries, shampoo formulations containing anionic surfactants and having long-term stability which at the same time exhibit excellent wet combabilities even without the use of silicones.

These objects were achieved by compositions comprising
a) at least one polymer a) with cationic and/or cationogenic groups and a molecular weight M_{w} in the range from 10 000 to 5 million and
b) at least one polymer b) selected from
   b1) polymers prepared in the presence of polymer a) which comprise a compound of the general formula I in copolymerized form

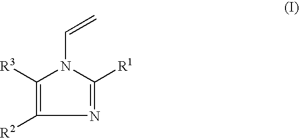

where R¹ to R³, independently of one another, are hydrogen, C₁-C₄-alkyl or phenyl, and
b2) cationic polymers prepared in the presence of polymer a) and at least one salt and comprising, in copolymerized form, at least one cationic monomer and at least one compound of the general formula I.
   Polymer a) Having Cationic and/or Cationogenic Groups

Cationogenic groups are understood as atomic groups covalently bonded in the polymer which can be converted by chemical reactions known to the person skilled in the art into a cationic charge state, i.e. into cationic groups.

Suitable polymers a) comprise at least one olefinically unsaturated, free-radically polymerizable compound with at least one cationic and/or cationogenic group per molecule in copolymerized form.

Preferably, the cationogenic and/or cationic groups are nitrogen-containing groups, such as primary, secondary and tertiary amino groups, and quaternary ammonium groups. Preferably, the nitrogen-containing groups are tertiary amino groups or quaternary ammonium groups. Charged cationic groups can be produced from the amine nitrogens either by protonation or by quaternization with acids or alkylating agents.

These include, for example, carboxylic acids, such as lactic acid, or mineral acids, such as phosphoric acid, sulfuric acid and hydrochloric acid, or as alkylating agents C₁-C₄-alkyl halides or sulfates, such as ethyl chloride, ethyl bromide, methyl chloride, methyl bromide, dimethyl sulfate and diethyl sulfate.

Preference is given to the olefinically unsaturated, free-radically polymerizable compounds having cationic/cationogenic groups selected from the group consisting of
esters of α,β-ethylenically unsaturated mono- and dicarboxylic acids with amino alcohols which may be mono- or dialkylated on the amine nitrogen,
amides of α,β-ethylenically unsaturated mono- and dicarboxylic acids with diamines which have at least one primary or secondary amino group,
N,N-diallylamine and derivatives thereof,
vinyl- and allyl-substituted nitrogen heterocycles,
vinyl- and allyl-substituted heteroaromatic compounds and
mixtures thereof.

Esters of α,β-ethylenically unsaturated mono- and dicarboxylic acids with amino alcohols which may be mono- or dialkylated on the amine nitrogen:

Preferred amino alcohols are C₂-C₁₂-amino alcohols which are C₁-C₈-mono- or -dialkylated on the amine nitrogen. Suitable acid components of these esters are, for example, acrylic acid, methacrylic acid, fumaric acid, maleic acid, itaconic acid, crotonic acid, maleic anhydride, monobutyl maleate and mixtures thereof. Preference is given to using acrylic acid, methacrylic acid and mixtures thereof. As compound c), particular preference is given to using N-methylaminoethyl (meth)acrylate, N-ethylaminoethyl (meth)acrylate, N-(n-propyl)aminoethyl (meth)acrylate, N-(n-butyl)aminoethyl (meth)acrylate, N-(tert-butyl)aminoethyl (meth)acrylate, N,N-dimethylaminomethyl (meth)acrylate, N,N-dimethylaminoethyl (meth)acrylate, N,N-diethylaminoethyl (meth)acrylate, N,N-dimethylaminopropyl (meth)acrylate, N,N-diethylaminopropyl (meth)acrylate and N,N-dimethylaminocyclohexyl (meth)acrylate. In particular, N-(tert-butyl)aminoethyl acrylate and N-(tert-butyl)aminoethyl methacrylate are used.

Amides of α,β-ethylenically unsaturated mono- and dicarboxylic acids with diamines which have at least one primary or secondary amino group:

Preference is given to diamines which have one tertiary and one primary or secondary amino group. As such monomers, preference is given to using N-[2-(dimethyl-amino)ethyl]acrylamide, N-[2-(dimethylamino)ethyl]methacrylamide, N-[3-(dimethyl-amino)propyl]acrylamide, N-[3-(dimethylamino)propyl]methacrylamide, N-[4-(dimethyl-amino)butyl]acrylamide, N-[4-(dimethylamino)butyl]methacrylamide, N-[2-(diethyl-amino)ethyl]acrylamide, N-[4-(dimethylamino)cyclohexyl]acrylamide and N-[4-(dimethylamino)cyclohexyl]methacrylamide, where N-[3-(dimethylamino)propyl]-methacrylamide is particularly preferred.

Suitable N,N-diallylamines and derivatives thereof are, for example, N-alkyl derivatives and the corresponding acid addition salts; alkyl here is preferably C₁-C₂₄-alkyl.

Preference is given, for example, to N,N-diallyl-N-methylamine and diallyldimethylammonium chloride.

### Vinyl- and Allyl-Substituted Nitrogen Heterocycles:

Suitable examples thereof are N-vinylimidazole, N-vinylimidazole derivatives, for example N-vinyl-2-methylimidazole, vinyl- and allyl-substituted heteroaromatic compounds, such as 2- and 4-vinylpyridine, 2- and 4-allylpyridine, and the salts thereof. In particular, N-vinylimidazoles of the general formula (I), as defined later under polymer b), are suitable.

To produce a cationic charge of polymer a), the abovementioned monomers must either be used for the polymerization in an already quaternized state, or the monomers have to be at least partially quaternized by suitable reactions following production of polymer a) in the copolymerized state.

### Quaternization

The degree of quaternization of polymer a) (mol % of quaternized groups of all quaternizable groups) is at least 60%, preferably at least 70%, particularly preferably at least 80% and in particular at least 90%. Very particular preference is given to quaternizing all quaternizable groups, i.e. a degree of quaternization 100 mol %, based on the quaternizable groups.

Of suitability for the quaternization of the polymers a) and, if appropriate, b) are, for example, alkyl halides having 1 to 24 carbon atoms in the alkyl group, e.g. methyl chloride, methyl bromide, methyl iodide, ethyl chloride, ethyl bromide, propyl chloride, hexyl chloride, dodecyl chloride, lauryl chloride, propyl bromide, hexyl bromide, dodecyl bromide, lauryl bromide and benzyl halides, in particular benzyl chloride and benzyl bromide. For the quaternization with long-chain alkyl radicals, the corresponding alkyl bromides, such as hexyl bromide, dodecyl bromide or lauryl bromide are preferred. Further suitable quaternizing agents are dialkyl sulfates, in particular dimethyl sulfate or diethyl sulfate.

Preferred quaternizing agents are methyl chloride, dimethyl sulfate or diethyl sulfate, where methyl chloride and dimethyl sulfate are particularly preferred.

The quaternization of the monomers or polymers with one of the specified quaternizing agents takes place by customary methods known to the person skilled in the art.

Examples of polymers a) suitable according to the invention are explicitly described in WO 02/083073, paragraphs [0054] to [0056], to which reference is made in its entirety. Also suitable according to the invention are the polymers a) described in U.S. Pat. No. 6,110,451, column 7, line 46 to column 9, line 38, to which reference is likewise made in its entirety.

Also suitable according to the invention are the polyquaternium-1 to polyquaternium-71 described by the CTFA (Cosmetic, Toiletry, and Fragrance Association, 1101 17th Street, NW Suite 300 Washington, D.C. 20036-4702).

In this regard, reference is also made to WO 02/083073, [0056] in its entirety.

Preferred polymers a) comprise diallyldimethylammonium chloride (DADMAC) in copolymerized form. Preferably, the polymers a) comprise at least 30% by weight, particularly preferably at least 50% by weight, further preferably at least 70% by weight and particularly preferably at least 90% by weight, of DADMAC in copolymerized form.

In a preferred embodiment, polymer a) is a DADMAC homopolymer, such as, for example, polyquaternium-6. Polyquaternium-6 is available under the trade names AEC®Polyquaternium-6 (A & E Cannock (Perfumery & Cosmetics) Ltd.), Agequat®400 (CPS Chemical Company), Conditioner®P6 (3V Group), Flocare®C106 (SNF S.A.), Genamin®PDAC (Clariant GmbH), Mackernium®006 (McIntyre Group Ltd), Merquat®100 (Nalco Company), Merquat®106 (Nalco Company), Mirapol®100 (Rhodia Inc.), Octacare®PQ6 (The Associated Octel Company Ltd), Rheocare®CC6 (Cosmetic Rheologies, Ltd.), Rheocare®CC6P (Cosmetic Rheologies, Ltd.), Ritaquta 6 (Rita Corporation), Salcare®SC30 (Ciba Specialty Chemicals Corporation), Tinocare®PQ-6H (Ciba Specialty Chemicals Corporation) or Catiofast®CS (BASF).

Polymer a) preferably has a mass-average molecular weight M_{w} in the range from 10 000-2 000 000, preferably in the range from 50 000 to 500 000, particularly preferably in the range from 100 000 to 200 000 g/mol.

Polymer b1)

Polymer b1) is prepared in the presence of polymer a) and comprises a compound of the general formula I in copolymerized form

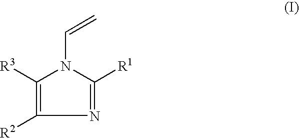

where R¹ to R³, independently of one another, are hydrogen, C₁-C₄-alkyl or phenyl. Preferably, the mass-average molecular weight M_{w} of polymer b1) is in the range from 1500 to 500 000, particularly preferably from 10 000 to 100 000 and in particular from 30 000 to 70 000 g/mol.

The amount of copolymerized compound of the general formula I is preferably from 10 to 100% by weight, further preferably from 20 to 70% by weight, particularly preferably from 30 to 60% by weight and in particular 40 to 60% by weight.

Examples of compounds of the general formula (I) can be found in the table below:

| R¹ | R² | R³ |
|---|---|---|
| H | H | H |
| Me | H | H |
| H | Me | H |
| H | H | Me |
| Me | Me | H |
| H | Me | Me |
| Me | H | Me |
| Ph | H | H |
| H | Ph | H |
| H | H | Ph |
| Ph | Me | H |
| Ph | H | Me |
| Me | Ph | H |
| H | Ph | Me |
| H | Me | Ph |
| Me | H | Ph |

Me = methyl
Ph = phenyl

Particular preference is given to N-vinylimidazole (NVI), i.e. the compound of the formula I, where all of the radicals R¹ to R³ are hydrogen.

Within the scope of this invention, if mass-average molecular weights M_{w} are stated, these are determined and/or to be determined by customary measurement methods known to the person skilled in the art. Preferred measurement methods for determining M_{w} are gel permeation chromatography (GPC) and field flow fractionation (FFF). The person skilled in the art knows what measurement conditions are to be used for which polymers.

In a preferred embodiment, the copolymerized NVI is present to at least 80 mol %, particularly preferably to at least 90 mol %, very particularly preferably to at least 95 mol % and in particular to at least 99 mol % in nonquaternized state. Most preferably, the copolymerized NVI is present to 100 mol % in nonquaternized state.

In a preferred embodiment of the invention, polymer b1) comprises, in copolymerized form,

i) in the range from 10 to 100% by weight, at least one compound of the general formula I and
ii) in the range from 0 to 90% by weight, at least one compound of the general formula II

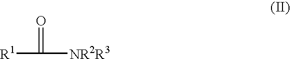

where R¹ is a group of the formula CH₂═CR⁴- where R⁴═H or C₁-C₄-alkyl, and R² and R³, independently of one another, are H, alkyl, cycloalkyl, heterocycloalkyl, aryl or hetaryl, or R² and R³, together with the nitrogen atom to which they are bonded, are a five- to eight-membered nitrogen heterocycle or

R² is a group of the formula CH₂═CR⁴—, and R¹ and R³, independently of one another, are H, alkyl, cycloalkyl, heterocycloalkyl, aryl or heteraryl, or R¹ and R³, together with the amide group to which they are bonded, are a lactam having 5 to 8 ring atoms.

Suitable compounds of the formula II are selected from primary amides, α,β-ethylenically unsaturated monocarboxylic acids, N-vinylamides of saturated monocarboxylic acids, N-vinyllactams, N-alkyl- and N,N-dialkylamides, α,β-ethylenically unsaturated monocarboxylic acids and mixtures thereof.

Suitable N-alkyl- and N,N-dialkylamides of α,β-ethylenically unsaturated monocarboxylic acids which, in addition to the carbonyl carbon atom of the amide group, have at most 8 further carbon atoms are, for example, N-methyl(meth)acrylamide, N-ethyl(meth)acrylamide, N-propyl(meth)acrylamide, N-(n-butyl)(meth)acrylamide, N-tert-butyl(meth)acrylamide, N,N-dimethyl(meth)acrylamide, N,N-diethyl(meth)acrylamide, piperidinyl(meth)acrylamide, morpholinyl(meth)acrylamide and mixtures thereof.

Polymer b1) particularly preferably comprises at least one N-vinyllactam in copolymerized form. Suitable N-vinyllactams are, for example, unsubstituted N-vinyl-lactams and N-vinyllactam derivatives which can have, for example, one or more C₁-C₆-alkyl substituents, such as methyl, ethyl, n-propyl, isopropyl, n-butyl, sec-butyl, tert-butyl etc. These include, for example, N-vinylpyrrolidone, N-vinylpiperidone, N-vinyl-caprolactam, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam, N-vinyl-7-ethyl-2-caprolactam, and mixtures thereof.

In particular, polymer b1) comprises, in copolymerized form, those compounds in which, in formula II, R² is CH₂═CH—, and R¹ and R³, together with the amide group to which they are bonded, are a lactam with 5 ring atoms.

Polymer b1) particularly preferably comprises, in copolymerized form, those compounds which are selected from acrylamide, methacrylamide, N-vinylpyrrolidone, N-vinylcaprolactam, N-vinylformamide, N-vinylacetamide and mixtures thereof. N-vinylpyrrolidone is most preferred.

Polymer b2)

Polymers b2) are cationic polymers prepared in the presence of polymer a) and at least one salt which comprise at least one cationic monomer and at least one compound of the abovementioned general formula I in copolymerized form. Suitable cationic monomers are described in detail above under polymer a).

The carrying-out of the water-in-water emulsion polymerization is known to the person skilled in the art for example from the published patent applications DE 10334262, DE 10338828, WO 2005/012378, WO 2006/018113, WO 98/54234 and DE 102005023799.

In particular, reference may be made to the details in WO 98/54234. This describes the preparation of aqueous polyvinylformamide dispersions by polymerization of vinyl-formamide in the presence of a water-soluble polymeric stabilizer, such as, for example, polyDADMAC and a water-soluble salt. For the description of the preparation of polymer b2) in the presence of polymer a), reference may be made to the disclosure of WO 98/54234 and EP 984990, to which reference is made in their entirety.

Example 8 of the present invention describes, by way of representation, a typical procedure for the preparation of a polymer b2) by a water-in-water emulsion polymerization in the presence of polymer a) and a salt.

Suitable salts are described in detail in the specifications WO 98/14405 and WO 00/20470, to which reference is hereby made in their entirety.

Suitable salts are accordingly inorganic salts, such as fluorides, chlorides, sulfates, phosphates or hydrogenphosphates of metal ions or ammonium ions. Typical representatives are sodium sulfate, potassium sulfate, ammonium sulfate, magnesium sulfate, aluminum sulfate, sodium chloride, calcium chloride, sodium dihydrogen-phosphate, diammonium hydrogenphosphate, dipotassium hydrogenphosphate, calcium phosphate, sodium citrate and iron sulfate.

Chaotropic salts, such as, for example, thiocyanates, perchlorates, chlorates, nitrates, bromides and iodides, can likewise be used. Typical representatives are calcium nitrate, sodium nitrate, ammonium nitrate, aluminum nitrate, sodium thiocyanate and sodium iodide.

Salts of organic C₁- to C₁₅-carboxylic acids, in particular the alkali metal salts, for example sodium or potassium salts or ammonium salts of mono-, di- or polybasic organic C₁- to C₁₂-carboxylic acids, such as, for example, formic acid, acetic acid, citric acid, oxalic acid, malonic acid, succinic acid, adipic acid, suberic acid, phthalic acid, agaric acid, trimesic acid, 1,2,3-propanetricarboxylic acid, and 1,4-, 2,3- or 2,6-naphthalenedicarboxylic acid are advantageously used.

Particular preference is given to using citrates, in particular sodium citrate and ammonium citrate.

The abovementioned salts can be used individually or as mixtures of two or more salts. Often, a mixture of two or more salts is more effective than one salt on its own, based on the amount used.

The salts are added in an amount which is 1 to 100% by weight, preferably 10 to 90% by weight and particularly preferably 15 to 75% by weight, of the saturation amount in the aqueous reaction medium under reaction conditions.

100% by weight saturation amount in the reaction medium is to be understood as meaning the amount of salt or salts which just still dissolves in the aqueous reaction medium of the monomers used in the presence of polymer a) and, if appropriate, further auxiliaries at the reaction temperature used, without precipitating.

According to the invention, the total amount of the at least one salt can be initially introduced in the reaction medium. However, it is also possible, if appropriate, to initially introduce merely some of the at least one salt in the reaction medium, and to introduce the, if appropriate, remaining amount or the total amount of the at least one salt to the reaction medium under polymerization conditions. However, it is to be ensured here that both the monomers used for the polymerization (until their complete reaction) and also the polymer b2) formed under reaction conditions in the aqueous reaction medium are always present as separate heterogeneous phase.

It is also of importance that, for the preparation of polymer b2), polymer a) can optionally also be used in combination with so-called neutral protective colloids known to the person skilled in the art, such as, for example, polyvinyl alcohols, poly-N-vinyl-2-pyrrolidone, polyalkylene glycols, and cellulose, starch or gelatin derivatives. However, in this connection, the weight fraction of optionally used neutral protective colloids is generally lower than the weight fraction of polymer a) and is often <5% by weight, <3% by weight or <1% by weight, in each case based on the total amount of the monomers used for the polymerization for polymer b2).

According to the invention, the total amount of polymer a) can be initially introduced if appropriate in combination with the neutral protective colloids in the reaction medium. However, it is also possible to initially introduce if appropriate merely some of polymer a), if appropriate in combination with the neutral protective colloids, in the reaction medium, and to introduce the, if appropriate, remaining amount or the total amount of polymer a), if appropriate in combination with the neutral protective colloids to the reaction medium under polymerization conditions.

### Further Monomers

The polymers a) and b) can comprise further monomers in copolymerized form. Examples of compounds suitable for the copolymerization are given below: monoethylenically unsaturated mono- and dicarboxylic acids having 3 to 25, preferably 3 to 6, carbon atoms, which can also be used in the form of their salts or anhydrides, such as, for example, acrylic acid, methacrylic acid, ethacrylic acid, α-chloroacrylic acid, crotonic acid, maleic acid, maleic anhydride, itaconic acid, citraconic acid, mesaconic acid, glutaconic acid, aconitic acid and fumaric acid;

esters of (meth)acrylic or ethacrylic acid, such as, for example, methyl (meth)acrylate, ethyl (meth)acrylate, n- and isopropyl (meth)acrylate, methyl ethacrylate, ethyl ethacrylate, n-propyl ethacrylate, isopropyl ethacrylate, n-butyl ethacrylate, tent-butyl ethacrylate, isobutyl ethacrylate, n-butyl (meth)acrylate, tert-butyl (meth)acrylate, isobutyl (meth)acrylate, sec-butyl (meth)acrylate, 2-pentyl (meth)acrylate, 3-pentyl (meth)acrylate, isopentyl acrylate, neopentyl acrylate, n-octyl (meth)acrylate, 1,1,3,3-tetramethylbutyl (meth)acrylate, ethylhexyl (meth)acrylate, n-nonyl (meth)acrylate, n-decyl (meth)acrylate, n-undecyl (meth)acrylate, tridecyl (meth)acrylate, myristyl (meth)acrylate, pentadecyl (meth)acrylate, palmityl (meth)acrylate, heptadecyl (meth)acrylate, nonadecyl (meth)acrylate, arachinyl (meth)acrylate, behenyl (meth)acrylate, lignocerenyi (meth)acrylate, cerotinyl (meth)acrylate, melissinyl (meth)acrylate, palmitoleinyi (meth)acrylate, oleyl (meth)acrylate, linolyl (meth)acrylate, linolenyl (meth)acrylate, stearyl (meth)acrylate, lauryl (meth)acrylate, phenoxyethyl acrylate, 4-t-butylcyclohexyl acrylate, cyclohexyl (meth)acrylate, ureido (meth)acrylate, tetrahydrofurfuryl (meth)acrylate and mixtures thereof;

alkyl vinyl ethers of the formula H₂C═CH—O—R, where R is C₁-C₃₀, preferably C₈- to C₂₂-alkyl, such as, for example, methyl vinyl ether, ethyl vinyl ether, n-propyl vinyl ether, isopropyl vinyl ether, n-butyl vinyl ether, sec-butyl vinyl ether, tert-butyl vinyl ether, dodecyl vinyl ether, octadecyl vinyl ether etc.;

amides of amino alcohols with olefinically unsaturated carboxylic acids, such as, for example, 2-hydroxypropylmethacrylamide, 3-hydroxypropylacrylamide, 3-hydroxy-propylmethacrylamide, 3-hydroxybutylacrylamide, 3-hydroxybutylmethacrylamide, 4-hydroxybutylacrylamide, 4-hydroxybutylmethacrylamide, 6-hydroxyhexylacrylamide, 6-hydroxyhexylmethacrylamide, 3-hydroxy-2-ethylhexylacrylamide and 3-hydroxy-2-ethylhexylmethacrylamide;

amides of amines with olefinically unsaturated carboxylic acids, such as, for example, N-(n-butyl)methacrylamide, N-(sec-butyl)methacrylamide, N-(tert-butyl)methacrylamide, N-(n-pentyl)(meth)acrylamide, N-(n-hexyl)(meth)acrylamide, N-(n-heptyl)(meth)acrylamide, N-(n-octyl)(meth)acrylamide, N-(tert-octyl)(meth)acrylamide, N-(1,1,3,3-tetramethylbutyl)(meth)acrylamide, N-ethylhexyl(meth)acrylamide, N-(n-nonyl)(meth)acrylamide, N-(n-decyl)(meth)acrylamide, N-(n-undecyl)(meth)acrylamide, N-tridecyl(meth)acrylamide, N-myristyl(meth)acrylamide, N-pentadecyl(meth)acrylamide, N-palmityl(meth)acrylamide, N-heptadecyl(meth)acrylamide, N-nonadecyl(meth)acrylamide, N-arachinyl(meth)acrylamide, N-behenyl(meth)acrylamide, N-lignocerenyl(meth)acrylamide, N-cerotinyl(meth)acrylamide, N-melissinyl(meth)acrylamide, N-palmitoleinyl(meth)acrylamide, N-oleyl(meth)acrylamide, N-linolyl(meth)acrylamide, N-linolenyl(meth)acrylamide, N-stearyl(meth)acrylamide, N-lauryl(meth)acrylamide;

vinyl esters, such as, for example, vinyl acetate, vinyl propionate, vinyl butyrate and mixtures thereof;

ethylene, propylene, isobutylene, butadiene, styrene, α-methylstyrene, acrylonitrile, methacrylonitrile, vinyl chloride, vinylidene chloride, vinyl fluoride, vinylidene fluoride and mixtures thereof.

Compounds suitable for the copolymerization which comprise C₈-C₃₀-, preferably C₈-C₂₂- and particularly preferably C₁₂-C₂₂-alkyl radicals are, for example, hexadecyl (meth)acrylate, octadecyl (meth)acrylate, hexadecyl vinyl ether or octadecyl vinyl ether. Compounds suitable for the copolymerization which comprise C₁-C₇-alkyl radicals are, for example, methyl (meth)acrylate, ethyl (meth)acrylate, n- and isopropyl (meth)acrylate, methyl ethacrylate, ethyl ethacrylate, n-propyl ethacrylate, isopropyl ethacrylate, n-butyl ethacrylate, tert-butyl ethacrylate, isobutyl ethacrylate, n-butyl (meth)acrylate, tert-butyl (meth)acrylate, isobutyl (meth)acrylate, sec-butyl (meth)acrylate, 2-pentyl (meth)acrylate, 3-pentyl (meth)acrylate, isopentyl acrylate, neopentyl acrylate, C₁-C₇-alkyl vinyl ethers e2) and mixtures thereof.

The abovementioned compounds suitable for the copolymerization are used in an amount of 0% by weight, preferably at least 0.3% by weight, particularly preferably at least 0.5% by weight and in particular at least 1% by weight and at most 30% by weight, preferably at most 20% by weight, particularly preferably at most 15% by weight and in particular at most 12% by weight, based on the total weight of the monomers used for the polymerization.

Most preferably, these compounds are used for the polymerization in an amount of from 1 to 10% by weight, based on the total weight of the monomers used for the polymerization.

The polymers a) and b) can also comprise at least one olefinically unsaturated, free-radically polymerizable compound with at least one anionogenic and/or anionic group per molecule in copolymerized form.

Such compounds can be selected from monoethylenically unsaturated carboxylic acids, sulfonic acids, phosphonic acids and mixtures thereof.

These compounds include monoethylenically unsaturated mono- and dicarboxylic acids having 3 to 25, preferably 3 to 6, carbon atoms, which can also be used in the form of their salts or anhydrides. Examples thereof are acrylic acid, methacrylic acid, ethacrylic acid, α-chloroacrylic acid, crotonic acid, maleic acid, maleic anhydride, itaconic acid, citraconic acid, mesaconic acid, glutaconic acid, aconitic acid and fumaric acid. These compounds also include the half-esters of monoethylenically unsaturated dicarboxylic acids having 4 to 10, preferably 4 to 6, carbon atoms, e.g. of maleic acid, such as monomethyl maleate. These compounds also include monoethylenically unsaturated sulfonic acids and phosphonic acids, for example vinylsulfonic acid, allylsulfonic acid, sulfoethyl acrylate, sulfoethyl methacrylate, sulfopropyl acrylate, sulfopropyl methacrylate, 2-hydroxy-3-acryloxypropylsulfonic acid, 2-hydroxy-3-methacryloxypropylsulfonic acid, styrenesulfonic acid, 2-acrylamido-2-methylpropanesulfonic acid, vinylphosphonic acid and allylphosphonic acid. These compounds also include the salts of the abovementioned acids, in particular the sodium, potassium and ammonium salts, and the salts with amines. These compounds can be used as such or as mixtures with one another. The stated weight fractions all refer to the acid form. If such an anionic or anionogenic compound is to be used, then it is preferably selected from the group consisting of acrylic acid, methacrylic acid, ethacrylic acid, α-chloroacrylic acid, crotonic acid, maleic acid, maleic anhydride, fumaric acid, itaconic acid, citraconic acid, mesaconic acid, glutaconic acid, aconitic acid and mixtures thereof, particularly preferably selected from acrylic acid, methacrylic acid, itaconic acid and mixtures thereof.

### Polymerization

To prepare the polymers b) in the presence of polymer a), the mixture to be polymerized can be polymerized both with the help of initiators that form free radicals, and through the action of high-energy radiation, under which should also be understood the action of high-energy electrons.

Initiators for the free-radical polymerization which can be used are the peroxo and/or azo compounds customary for this purpose, for example alkali metal or ammonium peroxydisulfates, diacetyl peroxide, dibenzoyl peroxide, succinyl peroxide, di-tert-butyl peroxide, tert-butyl perbenzoate, tert-butyl perpivalate, Cert-butyl peroxy-2-ethylhexanoate, tert-butyl permaleate, cumene hydroperoxide, diisopropyl peroxydicarbamate, bis(o-toluoyl) peroxide, didecanoyl peroxide, dioctanoyl peroxide, dilauroyl peroxide, Cert-butyl perisobutyrate, Cert-butyl peracetate, di-tert-amyl peroxide, tert-butyl hydroperoxide, azobisisobutyronitrile, azobis(2-amidonopropane) dihydrochloride or 2,2′-azobis(2-methylbutyronitrile).

Also suitable are initiator mixtures or redox initiator systems, such as, for example, ascorbic acid/iron(II) sulfate/sodium peroxodisulfate, tert-butyl hydroperoxide/sodium disulfite, tert-butyl hydroperoxide/sodium hydroxymethanesulfinate.

Preferably, organic peroxides are used.

The polymerization can also be carried out through the action of ultraviolet radiation, if appropriate in the presence of UV initiators. For polymerization under the action of UV rays, the photoinitiators or sensitizers customarily suitable for this purpose are used. These are, for example, compounds such as benzoin and benzoin ether, α-methylbenzoin or α-phenylbenzoin. So-called triplet sensitizers, such as benzyl diketals, can also be used. Serving as UV radiation sources are, for example, besides high-energy UV lamps, such as carbon arc lamps, mercury vapor lamps or xenon lamps, also low-UV light sources, such as fluorescent tubes with a high blue fraction.

The amounts of initiator or initiator mixtures used, based on monomer used, are between 0.01 and 10% by weight, preferably between 0.1 and 8% by weight, in particular between 1 and 6% by weight.

The preparation of polymer b1) can be carried out, for example, as solution polymerization, emulsion polymerization, inverse emulsion polymerization, suspension polymerization, inverse suspension polymerization or precipitation polymerization, without the methods which can be used being limited thereto.

It is preferred to carry out the polymerization as free-radical solution polymerization. Suitable solvents are, for example, water, alcohols, such as methanol, ethanol, n-propanol, isopropanol, n-butanol, sec-butanol, tert-butanol, n-hexanol and cyclo-hexanol, and also glycols, such as ethylene glycol, propylene glycol and butylene glycol, and the methyl or ethyl ethers of the dihydric alcohols, diethylene glycol, triethylene glycol, glycerol, dioxane, butyl acetate, ethyl acetate and toluene, where water, alcohols and mixtures thereof are particularly preferred. In particular, the solvent used is water or an aqueous-ethanolic mixture.

The solids content, i.e. the total amount of the substances other than the solvent present in the polymerization solution, based on the total solution, is usually in the range from 10 to 40% by weight, preferably in the range from 20 to 30% by weight.

The polymerization takes place in the temperature range from 30 to 100° C., preferably in the range from 50 to 90° C. It is usually carried out under atmospheric pressure, but can also proceed under reduced or increased pressure, preferably between 1 and 5 bar.

The polymerization can also be carried out semicontinuously by firstly introducing some, e.g. about 10%, of the mixture to be polymerized and initiator, heating the mixture to polymerization temperature and, after the polymerization has started, adding the remainder of the mixture to be polymerized according to the progress of the polymerization.

In one embodiment of the invention, the polymerization of polymer b) in the presence of polymer a) can also take place in such a way that the polymerization of polymer b) is carried out in a first step up to a conversion of 90% by weight, preferably of 95% by weight, further preferably of 99% by weight, particularly preferably of 99.9% by weight, based on the total amount of the monomers which are used for the polymerization of polymer b), in the absence of polymer a) and, in a second step, the reaction mixture resulting after the first step and comprising polymer b) and the remaining monomers is further polymerized in the presence of polymer a). At the start of the second step, the weight ratio of the amount of residual monomers from the preparation of polymer b) to the amount of polymer a) is preferably less than 1:100, further preferably less than 1:150, particularly preferably less than 1:200.

### Regulators

The preparation of the polymers b) can take place in the presence of at least one molecular weight regulator. Regulators are used preferably in a use amount of from 0.0005 to 5% by weight, particularly preferably from 0.001 to 2.5% by weight and in particular from 0.01 to 1.5% by weight, based on the total weight of the monomers.

Regulators is the term generally used to refer to compounds with high transfer constants. Regulators increase the rate of chain transfer reactions and thus bring about a reduction in the degree of polymerization of the resulting polymers without influencing the gross reaction rate.

In the case of the regulators, a distinction can be made between mono-, bi- or polyfunctional regulators, according to the number of functional groups in the molecule which can lead to one or more chain transfer reactions. Suitable regulators are described in detail, for example, by K. C. Berger and G. Brandrup in J. Brandrup, E. H. Immergut, Polymer Handbook, 3rd Edition, John Wiley & Sons, New York, 1989, pp. II/81-II/141.

Suitable regulators are, for example, aldehydes such as formaldehyde, acetaldehyde, propionaldehyde, n-butyraldehyde or isobutyraldehyde.

Furthermore, regulators which can also be used are: formic acid, its salts or esters, such as ammonium formate, 2,5-diphenyl-1-hexene, hydroxylammonium sulfate, and hydroxylammonium phosphate.

Further suitable regulators are halogen compounds, e.g. alkyl halides, such as tetrachloromethane, chloroform, bromotrichloromethane, bromoform, allyl bromide, and benzyl compounds, such as benzyl chloride or benzyl bromide.

Further suitable regulators are allyl compounds, such as, for example, allyl alcohol, functionalized allyl ethers, such as allyl ethoxylates, alkyl allyl ethers, or glyceryl monoallyl ethers.

Furthermore, alcohols, such as, for example, isopropanol, can also be used as regulator.

Preferably, the regulators used are compounds which comprise sulfur in bonded form.

Compounds of this type are, for example, inorganic hydrogensulfites, disulfites and dithionites or organic sulfides, disulfides, polysulfides, sulfoxides and sulfones. These include di-n-butyl sulfide, di-n-octyl sulfide, diphenyl sulfide, thiodiglycol, ethylthio-ethanol, diisopropyl disulfide, di-n-butyl disulfide, di-n-hexyl disulfide, diacetyl disulfide, diethanol sulfide, di-t-butyl trisulfide, dimethyl sulfoxide, dialkyl sulfide, dialkyl disulfide and/or diaryl sulfide.

Particular preference is given to organic compounds which comprise sulfur in bonded form.

Preferably, compounds used as polymerization regulators are thiols (compounds which obtain sulfur in the form of SH groups, also referred to as mercaptans). As regulators, preference is given to mono-, bi- and polyfunctional mercaptans, mercapto alcohols and/or mercaptocarboxylic acids.

Examples of these compounds are allyl thioglycolates, ethyl thioglycolate, cysteine, 2-mercaptoethanol, 1,3-mercaptopropanol, 3-mercaptopropane-1,2-diol, 1,4-mercaptobutanol, mercaptoacetic acid, 3-mercaptopropionic acid, mercaptosuccinic acid, thioglycerol, thioacetic acid, thiourea and alkyl mercaptans, such as n-butyl mercaptan, n-hexyl mercaptan or n-dodecyl mercaptan.

Particularly preferred thiols are cysteine, 2-mercaptoethanol, 1,3-mercaptopropanol, 3-mercaptopropane-1,2-diol, thioglycerol, thiourea.

Examples of bifunctional regulators which comprise two sulfur atoms in bonded form are bifunctional thiols, such as, for example, dimercaptopropanesulfonic acid (sodium salt), dimercaptosuccinic acid, dimercapto-1-propanol, dimercaptoethane, dimercaptopropane, dimercaptobutane, dimercaptopentane, dimercaptohexane, ethylene glycol bisthioglycolates and butanediol bisthioglycolate.

Examples of polyfunctional regulators are compounds which comprise more than two sulfur atoms in bonded form. Examples thereof are trifunctional and/or tetrafunctional mercaptans.

Preferred trifunctional regulators are trifunctional mercaptans, such as, for example, trimethylpropane tris(2-mercaptoethanate), trimethylolpropane tris(3-mercapto-propionate), trimethylolpropane tris(4-mercaptobutanate), trimethylolpropane tris(5-mercaptopentanate), trimethylolpropane tris(6-mercaptohexanate), trimethylolpropane tris(2-mercaptoacetate), glyceryl thioglycolate, glyceryl thiopropionate, glyceryl thioethylate, glyceryl thiobutanate, 1,1,1-propanetriyl tris(mercaptoacetate), 1,1,1-propanethyltris(mercaptoethanate), 1,1,1-propanetriyl tris(mercaptopropionate), 1,1,1-propanetriyl tris(mercaptobutanate), 2-hydroxymethyl-2-methyl-1,3-propanediol tris(mercaptoacetate), 2-hydroxymethyl-2-methyl-1,3-propanediol tris(mercapto-ethanate), 2-hydroxymethyl-2-methyl-1,3-propanediol tris(mercaptopropionate), 2-hydroxymethyl-2-methyl-1,3-propanediol tris(mercaptobutanate).

Particularly preferred trifunctional regulators are glyceryl thioglycolate, trimethylolpropane tris(2-mercaptoacetate), 2-hydroxymethyl-2-methyl-1,3-propanediol tris(mercaptoacetate).

Preferred tetrafunctional mercaptans are pentaerythritol tetrakis(2-mercaptoacetate), pentaerythritol tetrakis(2-mercaptoethanate), pentaerythritol tetrakis(3-mercapto-propionate), pentaerythritol tetrakis(4-mercaptobutanate), pentaerythritol tetrakis(5-mercaptopentanate), pentaerythritol tetrakis(6-mercaptohexanate).

Further suitable polyfunctional regulators are polyfunctional regulators Si compounds of the formulae

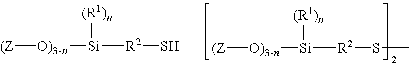

in which

n is a value from 0 to 2,

R¹ is a C₁-C₁₆-alkyl group or phenyl group,

R² is a C₁-C₁₈-alkyl group, the cyclohexyl group or phenyl group,

Z is a C₁-C₁₈-alkyl group, C₂-C₁₈-alkylene group or C₂-C₁₈-alkynyl group whose carbon atoms may be replaced by nonadjacent oxygen or halogen atoms, or is one of the groups

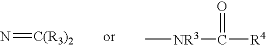

in which

R³/R³ is a C₁-C₁₂-alkyl group and

R⁴ is a C₁-C₁₈-alkyl group.

All of the specified regulators can be used individually or in combination with one another, where mercaptoethanol on its own or in mixtures is particularly preferred.

### Crosslinkers

In one embodiment of the invention, crosslinkers are used preparation of polymers b). The meaning of the term “crosslinker” is known to the person skilled in the art. The crosslinker is preferably selected from compounds with at least two ethylenically unsaturated, nonconjugated double bonds suitable for the free-radical copolymerization per molecule.

Suitable crosslinkers are, for example, acrylic esters, methacrylic esters, allyl ethers or vinyl ethers of at least dihydric alcohols. The OH groups of the parent alcohols may here be completely or partially etherified or esterified; however, the crosslinkers comprise at least two ethylenically unsaturated groups.

Examples of the parent alcohols are dihydric alcohols, such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentyl glycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, hydroxypivalic acid neopentyl glycol monoester, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and polyethylene glycols, polypropylene glycols and polytetra-hydrofurans with molecular weights of in each case 200 to 10 000.

Apart from the homopolymers of ethylene oxide and propylene oxide, it is also possible to use block copolymers of ethylene oxide or propylene oxide or copolymers which comprise ethylene oxide and propylene oxide groups in incorporated form.

Examples of parent alcohols having more than two OH groups are trimethylolpropane, glycerol, pentaerythritol, 1,2,5-pentanetriol, 1,2,6-hexanetriol, triethoxycyanuric acid, sorbitan, sugars, such as sucrose, glucose, mannose. Preferred polyhydric alcohols in this connection are also di- and trisaccharides.

The polyhydric alcohols can of course also be used following reaction with ethylene oxide or propylene oxide as the corresponding ethoxylates or propoxylates, respectively. The polyhydric alcohols can also firstly be converted to the corresponding glycidyl ethers by reaction with epichlorohydrin.

Further suitable crosslinkers are the vinyl esters or the esters of monohydric, unsaturated alcohols with ethylenically unsaturated C₃- to C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. However, it is also possible to esterify the monohydric, unsaturated alcohols with polybasic carboxylic acids, for example malonic acid, tartaric acid, trimellitic acid, phthalic acid, terephthalic acid, citric acid or succinic acid.

Further suitable crosslinkers are esters of unsaturated carboxylic acids with the above-described polyhydric alcohols, for example oleic acid, crotonic acid, cinnamic acid or 10-undecenoic acid.

Suitable crosslinkers are, furthermore, straight-chain or branched, linear or cyclic, aliphatic or aromatic hydrocarbons which have at least two double bonds which, in the case of aliphatic hydrocarbons, must not be conjugated, e.g. divinyibenzene, divinyltoluene, 1,7-octadiene, 1,9-decadiene, 4-vinyl-1-cyclohexene, trivinylcyclo-hexane or polybutadienes with molecular weights of from 200 to 20 000.

Also suitable as crosslinkers are the amides of (meth)acrylic acid, itaconic acid and maleic acid, and N-allylamines of at least difunctional amines. Such amines are, for example, 1,2-diaminomethane, 1,2-diaminoethane, 1,3-diaminopropane, 1,4-diamino-butane, 1,6-diaminohexane, 1,12-dodecanediamine, piperazine, diethylenetriamine or isophoronediamine. Likewise suitable are the amides of allylamine and unsaturated carboxylic acids, such as acrylic acid, methacyrlic acid, itaconic acid, maleic acid, or at least dibasic carboxylic acids, as have been described above.

Also suitable as crosslinkers are triallylamine and triallylmonoalkylammonium salts, e.g. triallylmethylammonium chloride or methyl sulfate.

Also suitable are N-vinyl or N-allyl compounds of urea derivatives, at least difunctional amides, cyanurates or urethanes, for example of urea, ethyleneurea, propyleneurea or tartardiamide, e.g. N,N′-divinylethyleneurea, N,N′-divinylpropyleneurea or N,N′-diallylurea.

Also suitable are alkylenebisacrylamides, such as methylenebisacrylamide and N,N′-(2,2)butane and 1,1′-bis(3,3′-vinylbenzimidazolith-2-one)-1,4-butane.

Other suitable crosslinkers are, for example, alkylene glycol di(meth)acrylates, such as ethylene glycol diacrylate, ethylene glycol dimethacrylate, tetraethylene glycol acrylate, tetraethylene glycol dimethacrylate, diethylene glycol acrylate, diethylene glycol methacrylate, vinyl acrylate, allyl acrylate, allyl methacrylate, divinyldioxane, penta-erythritol allyl ether, and mixtures of these crosslinkers.

Further suitable crosslinkers are divinyldioxane, tetraallylsilane or tetravinylsilane.

Crosslinkers that are particularly preferably used are methylenebisacrylamide, tri-allylamine, triallylalkylammonium salts, divinylimidazole, pentaerythritol triallyl ether, N,N′-divinylethyleneurea, reaction products of polyhydric alcohols with acrylic acid or methacrylic acid, methacrylic acid esters and acrylic acid esters of polyalkylene oxides or polyhydric alcohols which have been reacted with ethylene oxide and/or propylene oxide and/or epichlorohydrin.

Very particularly preferred crosslinkers are pentaerythritol triallyl ether, methylenebis-acrylamide, N,N′-divinylethyleneurea, triallylamine and triallylmonoalkylammonium salts, and acrylic acid esters of ethylene glycol, butanediol, trimethyloipropane or glycerol or acrylic acid esters of glycol, butanediol, trimethyloipropane or glycerol reacted with ethylene oxide and/or epichlorohydrin. Pentaerythritol triallyl ether is most preferred.

It is of course also possible to use mixtures of the abovementioned compounds. The crosslinker is preferably soluble in the reaction medium. If the solubility of the crosslinker in the reaction medium is low, then it can be dissolved in a monomer or in a monomer mixture, or else be metered in dissolved in a solvent which mixes with the reaction medium. Particular preference is given to those crosslinkers which are soluble in the monomer mixture.

If crosslinkers are used for the polymerization, then in amounts of at least 0.01% by weight, preferably at least 0.05% by weight, particularly preferably at least 0.1% by weight and at most 5% by weight, preferably at most 2% by weight and particularly preferably at most 1% by weight, based on the total amount of the monomers to be polymerized.

In a particularly preferred embodiment of the invention, pentaerythritol triallyl ether is used in an amount of from 0.1% by weight to 0.7% by weight, most preferably in an amount of from 0.3% by weight to 0.6% by weight.

The % by weight amount of the crosslinker refers to the total amount of the monomers used for the preparation of the polymer.

The reaction solutions and/or dispersions present after polymerization are stable at 50° C. over a period of at least one week, preferably at least one month, particularly preferably at least 3 months, further preferably at least 6 months and in particular at least 12 months, i.e. essentially no relatively extensive phase separation takes place.

Polymer b2) is generally prepared in such a way that, for the polymerization, >20% by weight, often >25% by weight and frequently >30% by weight, of the monomers to be used, based on the total amount of the resulting aqueous polymer dispersion, are used.

It is essential that the total amount of the monomer to be used is polymerized to a conversion of >90% by weight, often >95% by weight or frequently >98% by weight.

The preparation of polymer b2) can take place either in accordance with the so-called batch procedure where the entire amount of the monomers to be used is the initial charge, or in accordance with the so-called feed procedure.

If the polymerization takes place in a batch procedure, all of the components apart from the free-radical initiator are the initial charge in the polymerization reactor. The aqueous polymerization mixture is then heated with stirring to the polymerization temperature and then the free-radical initiator is added batchwise or continuously.

In a preferred embodiment, the method according to the invention is carried out by means of a feed procedure. Here, some or all of the reaction components are metered into the aqueous reaction medium in their entirety or partially, batchwise or continuously, together or in separate feeds.

Advantageously, at least some of the at least one salt and of polymer a), and, if appropriate, some of the at least one free-radical initiator and/or of the monomers to be used is initially introduced in the aqueous reaction medium with stirring and, under polymerization conditions, the, if appropriate, remaining amounts of the at least one organic or inorganic salt and polymer a), and the total amount or, if appropriate, remaining amount of the at least one free-radical initiator and/or the monomers to be used is metered in discontinuously or, in particular, continuously.

The water-in-water dispersions obtained in the polymerization can be subjected to a physical or chemical aftertreatment following the polymerization process.

### Cosmetic Compositions

The cosmetic compositions according to the invention can be in the form of aqueous or aqueous-alcoholic solutions, O/W and W/O emulsions, hydrodispersion formulations, solids-stabilized formulations, stick formulations, PIT formulations, in the form of creams, foams, sprays (pump spray or aerosol), gels, gel sprays, lotions, oils, oil gels or mousse, and accordingly be formulated with customary further auxiliaries.

Preferred cosmetic compositions for the purposes of the present invention are shampoos and haircare compositions. Accordingly, the invention also relates to compositions for the cleansing and/or care of the hair.

In particular, the invention relates to haircare compositions selected from the group consisting of pretreatment compositions, hair rinses, hair conditioners, hair balms, leave-on hair treatments, rinse-off hair treatments, hair tonics, pomades, styling creams, styling lotions, styling gels, end fluids, hot-oil treatments and foam treatments. Furthermore, the invention relates to cosmetic compositions which are selected from gel creams, hydroformulations, stick formulations, cosmetic oils and oil gels, mascara, self-tanning compositions, face care compositions, bodycare compositions, aftersun preparations, hair shaping compositions and hair-setting compositions.

Further cosmetic compositions according to the invention are skin cosmetic compositions, in particular those for the care of the skin. These are in particular in the form of W/O or O/W skin creams, day and night creams, eye creams, face creams, antiwrinkle creams, mimic creams, moisturizing creams, bleaching creams, vitamin creams, skin lotions, care lotions and moisturizing lotions.

Furthermore, the polymer combinations according to the invention are suitable as ingredients for hair cosmetic preparations, such as face tonics, face masks, deodorants and other cosmetic lotions and for use in decorative cosmetics, for example as concealing stick, stage makeup, in mascara and eyeshadows, lipsticks, kohl pencils, eyeliners, makeup, foundations, blushers and powders and eyebrow pencils.

Furthermore, the compositions according to the invention can be used in nose strips for pore cleansing, in antiacne compositions, repellents, shaving compositions, hair removal compositions, personal hygiene compositions, footcare compositions and in babycare.

Besides the polymer combinations obtainable according to the invention, the compositions comprise further cosmetically acceptable additives, such as, for example, emulsifiers and coemulsifiers, solvents, surfactants, oil bodies, preservatives, perfume oils, cosmetic care substances and active ingredients, such as AHA acids, fruit acids, ceramides, phytantriol, collagen, vitamins and provitamins, for example vitamin A, E and C, retinol, bisabolol, panthenol, natural and synthetic photoprotective agents, natural substances, opacifiers, solubility promoters, repellents, bleaches, colorants, tinting agents, tanning agents (e.g. dihydroxyacetone), micropigments, such as titanium oxide or zinc oxide, superfatting agents, pearlescent waxes, consistency regulators, thickeners, solubilizers, complexing agents, fats, waxes, silicone compounds, hydrotropes, dyes, stabilizers, pH regulators, reflectors, proteins and protein hydrolyzates (e.g. wheat, almond or pea proteins), ceramide, protein hydrolyzates, salts, gel formers, consistency regulators, silicones, humectants (e.g. 1,2-pentanediol), refitting agents, UV photoprotective filters and further customary additives. Furthermore, to establish the properties desired in each case, further polymers in particular may also be present.

Further cosmetic compositions according to the invention are bodycare compositions and also washing, showering and bathing preparations.

For the purposes of this invention, washing, showering and bathing preparations are understood as meaning soaps of liquid to gel-like consistency, such as transparent soaps, luxury soaps, deodorant soaps, cream soaps, baby soaps, skin protection soaps, abrasive soaps and syndets, pasty soaps, soft soaps and washing pastes, liquid washing, showering and bathing preparations, such as washing lotions, shower baths and shower gels, foam baths, oil baths and scrub preparations, shaving foams, shaving lotions and shaving creams.

Suitable further ingredients for these washing, showering and bathing preparations according to the invention are described below.

Besides the polymer combinations, the compositions according to the invention comprise further cosmetically acceptable additives, such as, for example, emulsifiers and coemulsifiers, solvents, surfactants, oil bodies, preservatives, perfume oils, cosmetic care substances and active ingredients, such as AHA acids, fruit acids, ceramides, phytantriol, collagen, vitamins and provitamins, for example vitamin A, E and C, retinol, bisabolol, panthenol, natural and synthetic photoprotective agents, natural substances, opacifiers, solubility promoters, repellents, bleaches, colorants, tinting agents, tanning agents (e.g. dihydroxyacetone), micropigments, such as titanium oxide or zinc oxide, superfatting agents, pearlescent waxes, consistency regulators, thickeners, solubulizers, complexing agents, fats, waxes, silicone compounds, hydrotropes, dyes, stabilizers, pH regulators, reflectors, proteins and protein hydrolyzates (e.g. wheat, almond or pea proteins), ceramide, protein hydrolyzates, salts, gel formers, consistency regulators, silicones, humectants (e.g. 1,2-pentanediol), refatting agents, UV photoprotective filters and further customary additives. Furthermore, to establish the properties desired in each case, further polymers in particular may also be present.

The cosmetic compositions according to the invention comprise the polymer combinations according to the invention in an amount of from 0.01 to 20% by weight, preferably from 0.05 to 5% by weight, particularly preferably 0.1 to 1.5% by weight, based on the weight of the composition.

In a preferred embodiment of the invention, the bodycare compositions, washing, showering and bathing preparations and shampoos and haircare compositions according to the invention further comprise at least one surfactant.

In a further preferred embodiment of the invention, the bodycare compositions, washing, showering and bathing preparations, shampoos and haircare compositions according to the invention comprise, besides the polymers, also at least one oil and/or fat phase and a surfactant.

### Surfactants

Surfactants which can be used are anionic, cationic, nonionic and/or amphoteric surfactants.

Advantageous washing-active anionic surfactants for the purposes of the present invention are
acylamino acids and salts thereof, such as acyl glutamates, in particular sodium acyl glutamate
sarcosinates, for example myristoyl sarcosine, TEA-lauroyl sarcosinate, sodium lauroyl sarcosinate and sodium cocoyl sarcosinate,

sulfonic acids and salts thereof, such as
acyl isethionates, for example sodium or ammonium cocoyl isethionate
sulfosuccinates, for example dioctyl sodium sulfosuccinate, disodium laureth sulfosuccinate, disodium lauryl sulfosuccinate and disodium undecyleneamido MEA sulfosuccinate, disodium PEG-5 lauryl citrate sulfosuccinate and derivatives,
alkyl ether sulfates, for example sodium, ammonium, magnesium, MIPA, TIPA laureth sulfate, sodium myreth sulfate and sodium C₁₂₋₁₃ pareth sulfate,
alkyl sulfates, for example sodium, ammonium and TEA lauryl sulfate.

Further advantageous anionic surfactants are
taurates, for example sodium lauroyl taurate and sodium methyl cocoyl taurate,
ether carboxylic acids, for example sodium laureth-13 carboxylate and sodium PEG-6 cocamide carboxylate, sodium PEG-7 olive oil carboxylate
phosphoric acid esters and salts, such as, for example, DEA oleth-10 phosphate and dilaureth-4 phosphate,
alkylsulfonates, for example sodium cocomonoglyceride sulfate, sodium C₁₂₋₁₄ olefin-sultanate, sodium lauryl sulfoacetate and magnesium PEG-3 cocamide sulfate,
acyl glutamates, such as di-TEA palmitoyl aspartate and sodium caprylic/capric glutamate,
acyl peptides, for example palmitoyl hydrolyzed milk protein, sodium cocoyl hydrolyzed soya protein and sodium/potassium cocoyl hydrolyzed collagen, and carboxylic acids and derivatives, such as, for example, lauric acid, aluminum stearate, magnesium alkanolate and zinc undecylenate, ester carboxylic acids, for example calcium stearoyl lactylate, laureth-6 citrate and sodium PEG-4 lauramide carboxylate
alkylarylsulfonates.

Advantageous washing-active cationic surfactants for the purposes of the present invention are quaternary surfactants. Quaternary surfactants comprise at least one N atom which is covalently bonded to four alkyl or aryl groups. Alkylbetaine, alkylamido-propylbetaine and alkylamidopropylhydroxysultaine, for example, are advantageous. Further advantageous cationic surfactants for the purposes of the present invention are also
alkylamines,
alkylimidazoles and
ethoxylated amines
   and in particular salts thereof.

Advantageous washing-active amphoteric surfactants for the purposes of the present invention are acyl/dialkylethylenediamines, for example sodium acyl amphoacetate, disodium acyl amphodipropionate, disodium alkyl amphodiacetate, sodium acyl amphohydroxypropylsulfonate, disodium acyl amphodiacetate, sodium acyl amphopropionate, and N-coconut-fatty acid-amidoethyl-N-hydroxyethyl glycinate sodium salts.

Further advantageous amphoteric surfactants are N-alkylamino acids, for example aminopropylalkylglutamide, alkylaminopropionic acid, sodium alkylimidodipropionate and lauroamphocarboxyglycinate.

Advantageous washing-active nonionic surfactants for the purposes of the present invention are
alkanolamides, such as cocamides MEA/DEA/MIPA,
esters which are formed by esterification of carboxylic acids with ethylene oxide, glycerol, sorbitan or other alcohols,
ethers, for example ethoxylated alcohols, ethoxylated lanolin, ethoxylated polysiloxanes, propoxylated POE ethers, alkyl polyglycosides, such as lauryl glucoside, decyl glycoside and cocoglycoside, glycosides with an HLB value of at least 20 (e.g. Belsil®SPG 128V (Wacker)).

Further advantageous nonionic surfactants are alcohols and amine oxides, such as cocoamidopropylamine oxide.

Preferred anionic, amphoteric and nonionic shampoo surfactants are specified, for example, in “Kosmetik and Hygiene von Kopf bis Fuβ” [Cosmetics and hygiene from head to toe], Ed. W. Umbach, 3rd Edition, Wiley-VCH, 2004, pp. 131-134, to which reference is made at this point in its entirety.

Among the alkyl ether sulfates, sodium alkyl ether sulfates based on di- or triethoxylated lauryl and myristyl alcohol, in particular, are preferred. They surpass the alkyl sulfates to a considerable degree with regard to the insensitivity toward water hardness, the ability to be thickened, low-temperature solubility and, in particular, skin and mucosa compatibility. They can also be used as the sole washing raw materials for shampoos. Lauryl ether sulfate has better foaming properties than myristyl ether sulfate, but is inferior to this in terms of mildness.

Alkyl ether carboxylates with average and particularly with relatively high belong to the mildest surfactants overall, but exhibit a poor foaming and viscosity behavior. They are often used in combination with alkyl ether sulfates and amphoteric surfactants in hair-washing compositions.

Sulfosuccinic acid esters (sulfosuccinates) are mild and readily foaming surfactants, but, on account of their poor ability to be thickened, are preferably only used together with other anionic and amphoteric surfactants and, on account of their low hydrolysis stability, are preferably only used in neutral or well buffered products.

Amidopropylbetaines are virtually insignificant as sole washing raw materials since their foaming behavior and their ability to be thickened are only moderately pronounced. By contrast, these surfactants have excellent skin and eye mucosa compatibility. In combination with anionic surfactants, their mildness can be synergistically improved. Preference is given to the use of cocamidopropylbetaine. Amphoacetates/amphodiacetates have, as amphoteric surfactants, very good skin and mucosa compatibility and can have a hair-conditioning effect and/or increase the care effect of additives. They are used similarly to the betaines for optimizing alkyl ether sulfate formulations. Sodium cocoamphoacetate and disodium cocoamphodiacetate are most preferred.

Alkyl polyglycosides are nonionic washing raw materials. They are mild, have good universal properties, but are weakly foaming. For this reason, they are preferably used in combination with anionic surfactants.

Sorbitan esters likewise belong to the nonionic washing raw materials. On account of their excellent mildness, they are preferably used in baby shampoos. As low-foamers, they are preferably used in combination with anionic surfactants.

It is advantageous to choose the washing-active surfactant(s) from the group of surfactants which have an HLB value of more than 25, those which have an HLB value of more than 35 being particularly advantageous.

According to the invention, it is advantageous if one or more of these surfactants are used in a concentration from 1 to 30% by weight, preferably in a concentration of from 5 to 25% by weight and very particularly preferably in a concentration of from 10 to 20% by weight, in each case based on the total weight of the composition.

### Polysorbates

Washing-active agents which can also advantageously be incorporated into the compositions according to the invention are polysorbates.

Polysorbates advantageous for the purposes of the invention are, for example,
polyoxyethylene(20) sorbitan monolaurate (Tween®20, CAS No. 9005-64-5)
polyoxyethylene(4) sorbitan monolaurate (Tween®21, CAS No. 9005-64-5)
polyoxyethylene(4) sorbitan monostearate (Tween®61, CAS No. 9005-67-8)
polyoxyethylene(20) sorbitan tristearate (Tween®65, CAS No. 9005-71-4)
polyoxyethylene(20) sorbitan monooleate (Tween®80, CAS No. 9005-65-6)
polyoxyethylene(5) sorbitan monooleate (Tween®81, CAS No. 9005-65-5)
polyoxyethylene(20) sorbitan trioleate (Tween®85, CAS No. 9005-70-3).
Polyoxyethylene(20) sorbitan monopalmitate (Tween®40, CAS No. 9005-66-7) and
polyoxyethylene(20) sorbitan monostearate (Tween®60, CAS No. 9005-67-8) are particularly advantageous.

The polysorbates are advantageously used in a concentration of from 0.1 to 5% by weight and in particular in a concentration of from 1.5 to 2.5% by weight, based on the total weight of the composition, individually or as a mixture of two or more polysorbates.

### Conditioning Agents

If desired, conditioning agents selected for the cosmetic compositions according to the invention are preferably those conditioning agents which are described on page 34, line 24 to page 37, line 10 of WO 2006/106140. Reference is hereby made to the content of the specified reference in its entirety.

The compositions according to the invention are suitable for intensifying and/or increasing the deposition amount and rate and also the residence time of further active ingredients optionally likewise present in these compositions according to invention, such as, for example, silicones or UV photoprotective filters, on the skin and/or the hair. Substances or agents which have such effects are also referred to as depositioning aids.

U.S. Pat. No. 6,998,113 describes rinse-off preparations which lead to the skin treated therewith being effectively protected against UV radiation. Some of the preparations described therein comprise cationic polymers. Within the context of the present invention, the combinations according to the invention of polymer a) and polymer b) can also be used in the preparations of U.S. Pat. No. 6,998,113. In particular, the combinations according to the invention can be used in sunscreen, washing and bathing preparations for the purpose specified by U.S. Pat. No. 6,998,113. Reference is hereby made to the disclosure of U.S. Pat. No. 6,998,113 in its entirety.

Suitable silicones are listed, for example, in U.S. Pat. No. 5,935,561, column 13, line 64 to column 18, line 61, to which reference is hereby made in its entirety.

By way of representation, mention may be made of:
dimethicones
polyalkyl- or polyarylsiloxanes (U.S. Pat. No. 5,935,561, column 13, formula I)
alkylamino-substituted silicones (U.S. Pat. No. 5,935,561, column 14, formula II (amodimethicones))
cationic silicones (U.S. Pat. No. 5,935,561, columns 14 and 15, formula III)
trimethylsilylamodimethicones (U.S. Pat. No. 5,935,561, column 15, formula IV)
silicones as in U.S. Pat. No. 5,935,561, column 15, formula V
cyclic polysiloxanes as in U.S. Pat. No. 5,935,561, column 16, formula VI.

### Rheology Modifiers

Suitable rheology modifiers are primarily thickeners.

Thickeners suitable for shampoos and haircare compositions are specified in “Kosmetik and Hygiene von Kopf bis Fuβ” [Cosmetics and hygiene from head to toe], Ed. W. Umbach, 3rd Edition, Wiley-VCH, 2004, pp. 235-236, to which reference is made at this point in its entirety.

Suitable thickeners for the cosmetic compositions according to the invention are described, for example, on page 37, line 12 to page 38, line 8 of WO 2006/106140. Reference is hereby made to the content of the specified reference in its entirety. Particular preference is given to cosmetic compositions according to the invention which comprise polyurethanes as so-called associative thickeners. Polyurethanes of this type are described, for example, in EP 1584331, EP 1013264, WO 2006/002813, EP 1241198, WO 02/083093, WO 02/088212, WO 02/044236, EP 725097, U.S. Pat. No. 4,079,028, EP 618243. Particular preference is given to cosmetic compositions according to the invention which comprise polyurethanes as described in EP 1584331, paragraphs [0009] to [0016]. Reference is hereby made to the disclosures of the abovementioned specifications in their entirety.

### Preservatives

The cosmetic compositions according to the invention can also comprise preservatives. Compositions with high water contents have to be reliably protected against the build-up of germs. Suitable preservatives for the cosmetic compositions according to the invention are described, for example, on page 38, line 10 to page 39, line 18 of WO 2006/106140. Reference is hereby made to the content of the specified reference in its entirety.

Complexing agents: Since the raw materials and also the shampoos themselves are prepared predominantly in steel apparatuses, the end products can comprise iron (ions) in trace amounts. In order to prevent these impurities adversely affecting the product quality via reactions with dyes and perfume oil constituents, complexing agents, such as salts of ethylenediaminetetraacetic acid, of nitrilotriacetic acid, of iminodisuccinic acid or phosphates are added.

UV photoprotective filters: In order to stabilize the ingredients present in the compositions according to the invention, such as, for example, dyes and perfume oils, against changes as a result of UV light, UV photoprotective filters, such as, for example, benzophenone derivatives, can be incorporated. Suitable UV photoprotective filters for the cosmetic compositions according to the invention are described, for example, on page 39, line 20 to page 41 line 10 of WO 2006/106140. Reference is hereby made to the content of the specified reference in its entirety.

Antioxidants: A content in the compositions according to the invention of antioxidants is generally preferred. According to the invention, antioxidants which can be used are all antioxidants which are customary or suitable for cosmetic applications. Suitable antioxidants for the cosmetic compositions according to the invention are described, for example, on page 41, line 12 to page 42 line 33 of WO 2006/106140. Reference is hereby made to the content of the specified reference in its entirety.

Buffers: Buffers ensure the pH stability of the compositions. By and large, citrate, lactate and phosphate buffers are used.

Solubility promoters: They are used in order to dissolve care oils or perfume oils to give clear solutions and also to maintain clear solutions at low temperature. The most common solubility promoters are ethoxylated nonionic surfactants, e.g. hydrogenated and ethoxylated castor oils.

Antimicrobial agents: Furthermore, antimicrobial agents can also be used. These include, in general, all suitable preservatives with a specific effect toward Gram-positive bacteria, e.g. triclosan (2,4,4′-trichloro-2′-hydroxydiphenyl ether), chlorhexidine (1,1′-hexamethylenebis[5-(4-chlorophenyl)biguanide), and TTC (3,4,4′-trichlorocarbanilide). Quaternary ammonium compounds are in principle likewise suitable and are preferably used for disinfecting soaps and washing lotions. Numerous fragrances also have antimicrobial properties. Also, a large number of essential oils or their characteristic ingredients, such as, for example, oil of cloves (eugenol), mint oil (menthol) or thyme oil (thymol), exhibit marked antimicrobial effectiveness.

The antibacterially effective substances are generally used in concentrations of from about 0.1 to 0.3% by weight.

Dispersants: if insoluble active ingredients, e.g. antidandruff active ingredients or silicone oils, are to be dispersed or kept permanently in suspension in the compositions according to the invention, dispersants and thickeners, such as, for example, magnesium aluminum silicates, bentonites, fatty acyl derivatives, polyvinylpyrrolidone or hydrocolloids, e.g. xanthan gum or carbomers, have to be used.

According to the invention, preservatives in a total concentration of at most 2% by weight, preferably at most 1.5% by weight and particularly preferably at most 1% by weight, based on the total weight of the composition, are present.

### Oils, Fats and Waxes

The compositions according to the invention preferably comprise oils, fats and/or waxes.

Advantageously, the ingredients chosen for the compositions according to the invention are those oils, fats and/or waxes which are described on page 28, line 39 to page 34, line 22 of WO 2006/106140. Reference is hereby made to the content of the specified reference in its entirety.

The content of further oils, fats and waxes is at most 50% by weight, preferably 30% by weight, further preferably at most 20% by weight, based on the total weight of the composition.

Apart from the abovementioned substances, the compositions can, if appropriate, comprise the additives customary in cosmetics, for example perfume, dyes, refatting agents, complexing and sequestering agents, pearlizing agents, plant extracts, vitamins, active ingredients, pigments which have a coloring effect, softening, moisturizing and/or humectant substances, or other customary constituents of a cosmetic or dermatological formulation, such as alcohols, polyols, polymers, organic acids for pH adjustment, foam stabilizers, electrolytes, organic solvents or silicone derivatives.

With regard to the specified further ingredients known to the person skilled in the art for the compositions, reference may be made to “Kosmetik and Hygiene von Kopf bis Fuβ” [Cosmetics and hygiene from head to toe], Ed. W. Umbach, 3rd Edition, Wiley-VCH, 2004, pp. 123-128, to which reference is made at this point in its entirety.

### Ethoxylated Glycerol Fatty Acid Esters

The compositions according to the invention such as bodycare compositions, washing and bathing preparations and shampoos and haircare compositions comprise, if appropriate, ethoxylated oils selected from the group of ethoxylated glycerol fatty acid esters, particularly preferably PEG-10 olive oil glycerides, PEG-11 avocado oil glycerides, PEG-11 cocoa butter glycerides, PEG-13 sunflower oil glycerides, PEG-15 glyceryl isostearate, PEG-9 coconut fatty acid glycerides, PEG-54 hydrogenated castor oil, PEG-7 hydrogenated castor oil, PEG-60 hydrogenated castor oil, jojoba oil ethoxylate (PEG-26 jojoba fatty acids, PEG-26 jojoba alcohol), glycereth-5 cocoate, PEG-9 coconut fatty acid glycerides, PEG-7 glyceryl cocoate, PEG-45 palm kernel oil glycerides, PEG-35 castor oil, olive oil PEG-7 ester, PEG-6 caprylic acid/capric acid glycerides, PEG-10 olive oil glycerides, PEG-13 sunflower oil glycerides, PEG-7 hydrogenated castor oil, hydrogenated palm kernel oil glyceride PEG-6 ester, PEG-20 corn oil glycerides, PEG-18 glyceryl oleate cocoate, PEG-40 hydrogenated castor oil, PEG-40 castor oil, PEG-60 hydrogenated castor oil, PEG-60 corn oil glycerides, PEG-54 hydrogenated castor oil, PEG-45 palm kernel oil glycerides, PEG-80 glyceryl cocoate, PEG-60 almond oil glycerides, PEG-60 evening primrose glycerides, PEG-200 hydrogenated glyceryl palmate, PEG-90 glyceryl isostearate.

Preferred ethoxylated oils are PEG-7 glyceryl cocoate, PEG-9 coconut glyceride, PEG-40 hydrogenated castor oil, PEG-200 hydrogenated glyceryl palmate.

Ethoxylated glycerol fatty acid esters are used in aqueous cleaning formulations for various purposes. Glycerol fatty acid esters with a degree of ethoxylation of about 30-50 serve as solubility promoters for nonpolar substances such as perfume oils. Highly ethoxylated glycerol fatty acid esters are used as thickeners.

### Active Ingredients

The most diverse of active ingredients with varying solubility can be incorporated homogeneously into the compositions according to the invention. Advantageous active ingredients in the cosmetic compositions according to the invention are described, for example, on page 44, line 24 to page 49, line 39 of WO 2006/106140. Reference is hereby made to the content of the specified reference in its entirety.

### UV Photoprotective Agents

In a preferred embodiment, the compositions according to the invention comprise UV photoprotective agents for protecting the skin and/or the hair. Suitable UV photoprotective agents are described in detail in WO 2006/106114, p. 24, I. 4 to p. 27, I. 27, to which reference is hereby made in its entirety.

The compositions advantageously comprise substances which absorb UV radiation in the UVB region and substances which absorb UV radiation in the UVA region, where the total amount of the filter substances is, for example, 0.1 to 30% by weight, preferably 0.5 to 20% by weight, in particular 1 to 15% by weight, based on the total weight of the compositions, in order to provide cosmetic compositions which protect the skin against the entire range of ultraviolet radiation.

The majority of the photoprotective agents in the cosmetic or dermatological compositions serving to protect the human epidermis consists of compounds which absorb UV light in the UV-B region. For example, the proportion of UV-A absorbers to be used according to the invention is 10 to 90% by weight, preferably 20 to 50% by weight, based on the total amount of substances absorbing UV-B and UV-A.

### Pearlescent Waxes

Suitable pearlescent waxes for the cosmetic compositions according to the invention are described, for example, on page 50, line 1 to line 16 of WO 2006/106140. Reference is hereby made to the content of the specified reference in its entirety. The compositions according to the invention can further comprise glitter substances and/or other effect substances (e.g. color streaks).

### Emulsifiers

In a preferred embodiment of the invention, the cosmetic compositions according to the invention are in the form of emulsions. The preparation of such emulsions takes place by known methods. Suitable emulsifiers according to the invention are described, for example, on page 50, line 18 to page 53, line 4 of WO 2006/106140. Reference is hereby made to the content of the specified reference in its entirety.

### Perfume Oils

If perfume oils are to be added to the cosmetic compositions according to the invention, then suitable perfume oils are described, for example, on page 53, line 10 to page 54, line 3 of WO 2006/106140. Reference is hereby made to the content of the specified reference in its entirety.

### Pigments

If appropriate, the cosmetic compositions according to the invention further comprise pigments. The pigments are present in the product mostly in undissolved form and may be present in an amount of from 0.01 to 25% by weight, particularly preferably from 5 to 15% by weight. The preferred particle size is 1 to 200 μm, in particular 3 to 150 μm, and particularly preferably 10 to 100 μm.

Suitable pigments for the compositions according to the invention are described, for example, on page 54, line 5 to page 55, line 19 of WO 2006/106140. Reference is hereby made to the content of the specified reference in its entirety.

### Polymers

In a preferred embodiment, the cosmetic compositions according to the invention comprise further polymers apart from the polymer prepared by the method according to the invention.

Suitable additional polymers for the compositions according to the invention are described, for example, on page 55, line 21 to page 63, line 2 of WO 2006/106140. Reference is hereby made to the content of the specified reference in its entirety.

### Shampoo Types

Depending on the hair quality or scalp problem, additional requirements are placed, if appropriate, on shampoos. The mode of action of the preferred shampoo types with the most important additional effects or most important specific objectives is described below.

According to the invention, preference is given, for example, to shampoos for normal or rapidly greasing or damaged hair, antidandruff shampoos, baby shampoos and 2-in-1 shampoos (shampoo and rinse in one).

Shampoos for normal hair: hair washing should free hair and scalp from the skin sebum formed in sebaceous glands, the inorganic salts emerging from sweat glands with water, amino acids, urea and lactic acid, flaked-off skin particles, environmental dirt, odors and, if appropriate, residues of hair cosmetic treatments. Normal hair means short to shoulder length hair which is only slightly damaged. Accordingly, the fraction of conditioning auxiliaries should be optimized to this hair type.

Shampoos for rapidly greasing hair: increased sebum production of the sebaceous glands in the scalp leads to a straggly, unattractive hairstyle just 1-2 days after hair washing. Oil and wax-like skin sebum constituents weigh down the hair and lower the friction from hair to hair and thus reduce the hairstyle hold. The actual hair cosmetic problem in the case of rapidly greasing hair is thus the premature collapse of voluminous hairstyles. In order to avoid this, it is necessary to prevent the surface of the hair becoming weighed down and too smooth and supple. This is preferably achieved through the surfactant base of washing raw materials that are good at cleaning and of particularly low substantivity. Additional care substances which would be added to the skin sebum, such as refitting substances or conditioning auxiliaries, are only used in shampoos for rapidly greasing hair with the greatest of care, if at all. Voluminizing shampoos for fine hair can be formulated comparably.

Shampoos for dry, stressed (damaged) hair. The structure of the hair in the course of hair growth is changed as a result of mechanical influences such as combing, brushing and especially back combing (combing against the direction of growth), as a result of the effect of UV radiation or visible light and as a result of cosmetic treatments, such as permanent waving, bleaching or coloring. The scale layer of the hair has an increasingly stressed appearance from the root to the end; in extreme cases, it is completely worn away at the end, and the hair ends are split (split ends). Damaged hair can in principle no longer be restored to the condition of healthy hair regrowth. However, it is possible to come very close to this ideal condition with regard to feel, shine and combability through use of shampoos according to the invention with, if appropriate, high fractions of care substances (conditioners).

An even better effect than with a shampoo is achieved with a haircare composition according to the invention for example in the form of a rinse or cure treatment after hair washing.

2-in-1 shampoos according to the invention are particularly strongly caring shampoos in which, through the conception as “shampoo and rinse in one”, the additional benefit of care is placed equally alongside the basic benefit of cleaning. 2-in-1 compositions according to the invention comprise increased amounts of conditioners.

Antidandruff shampoos: Compared with antidandruff hair tonics, antidandruff shampoos according to the invention have the advantage that they not only reduce the formation of new visible flakes through corresponding active ingredients to combat an attack of dandruff, and prevent such formation in the case of long-term use, but also remove flakes which have already flaked off with the hair washing. After rinsing out the wash liquor, however, only a small but adequate amount of the active ingredients remains on the scalp and hair. There are various antidandruff active ingredients which can be incorporated into the shampoo compositions according to the invention, such as, for example, zinc pyrithione, ketoconazole, elubiol, clotrimazole, climbazole or piroctone olamine. In addition, these substances have an effect that normalizes the flaking.

The basis of antidandruff shampoos largely corresponds to the formulation of shampoos for normal hair with a good cleaning effect.

Baby shampoos: in a preferred embodiment of the invention, the shampoo preparations according to the invention are baby shampoos. These are optimally skin and mucosa compatible. Combinations of washing raw materials with very good skin compatibility form the basis of these shampoos. Additional substances for further improving the skin and mucosa compatibility and the care properties are advantageously added, such as, for example, nonionic surfactants, protein hydrolyzates and panthenol or bisabolol. All necessary raw materials and auxiliaries, such as preservatives, perfume oils, dyes etc., are selected under the aspect of high compatibility and mildness.

Shampoos for dry scalp: in a further preferred embodiment of the invention, the shampoo preparations according to the invention are shampoos for dry scalp. The primary aim of these shampoos is to prevent the scalp from drying out, since dry scalp can lead to irritation, reddening and inflammation. As in the case of baby shampoos, combinations of washing raw materials with very good skin compatibility form the basis of these shampoos. In addition, if appropriate, refatting agents and humectants, such as, for example, glycerol or urea, can be used.

The shampoo compositions according to the invention can also be present as shampoo concentrates with increased surfactant contents of 20-30%. They are based on special washing raw material combinations and consistency regulators which ensure good distributability and spontaneous foaming ability, even in a small use amount. A particular advantage is, for example, the possibility of achieving the productivity of 200 ml of shampoo with a 100 ml bottle.

### Supply Form

It is advantageous if the compositions according to the invention are stored in a tube, a pot, a bottle or squeezable bottle and are applied from this. Accordingly, tubes, pots, bottles or squeezable bottles which comprise a composition according to the invention are also in accordance with the invention.

### EXAMPLES

The following abbreviations are used below:
dem.: completely demineralized
Q: quaternized
VP: N-vinylpyrrolidone
VI: N-vinylimidazole
VI*MeCl: vinylimidazole quaternized with methyl chloride
VI*DMS: vinylimidazole quaternized with dimethyl sulfate
DMAEMA: dimethylaminoethyl methacrylate
TMAEMC: 2-trimethylammonium methyl methacrylate chloride
DADMAC: diallyldimethylammonium chloride

### Example 1

58.5 g of VI were mixed with 465.79 g of Catiofast® CS (31% strength by weight aqueous solution of poly-DADMAC M_{w}=140 00 g/mol) and 58.5 g of VP. The mixture obtained was heated to 90° C. and stirred at 150 rpm. Feeds 1 and 2 were metered in over 2.5 hours. The reaction mixture was then cooled to 60°, feed 3 was added as a batch and feed 4 was metered in over 30 minutes. This reaction mixture was left to after-react for 1 hour at 60° C. 5 g of Phenonip® were then added and the mixture was left to cool to room temperature.

Feed 1:

356.2 g water

3.27 g Wako®V50

Feed 2:

1.3 g mercaptoethanol

46.80 g water

Feed 3:

2.39 g tert-butyl hydroperoxide (70% strength by weight solution)

Feed 4:

1.83 g sodium disulfite

28.17 g dem. water

The polymer combinations of examples 2 to 7 were prepared analogously to example 1. For the preparation of the combination according to the invention to example 6, mercaptoethanol was initially introduced. The respective weight ratios of the monomers are given in the table below.

### Example 8

64.10 g of sodium citrate were mixed with 500 g of Catiofast® CS (30% strength by weight aqueous poly-DADMAC solution). This mixture was adjusted to pH 6.8 with 0.89 g sulfuric acid (50% strength). 31 g of feed 1 were then added and the mixture was stirred at 180 rpm for 10 min. The mixture was then rendered inert for 1 hour with nitrogen and heated to 65° C. After reaching 65° C., 4 g of feed 2 were added and the mixture was polymerized for 15 min. Then, the remainder of feed 1 was metered in over 2 hours and, at the same time, 11 g of feed 2 were metered in over 3 hours.

The mixture was then left to polymerize for 4 hours at 65°. The reaction mixture was then heated to 70° C. and, after this temperature had been reached, the remaining 15 g of feed 2 were metered in over the course of 30 min. The mixture was then left to after polymerize for a further 1.5 hours at 70° C. The reaction mixture was then cooled to room temperature.

Feed 1:

18.75 g VP

37.5 g 50% strength by weight Quat 311 solution (DMAEMA quaternized with diethyl sulfate)

98.76 g dem. water

Feed 2:

29.44 g dem. water

0.56 g Wako®V50

The polymer combinations of examples 9 to 12 were prepared analogously to example 8. Example 13 corresponds to example 11 with regard to the monomer ratio, but the polymer combination of example 13 was prepared in a batch procedure and with a lower solids content.

| | | | | | 0.2% WS | | | | | |
|---|---|---|---|---|---|---|---|---|---|---|
| | | | | | slight | | | | Wet | Wet |
| | | | | | waxy feel | | | | combability | combability |
| | | | | | ++ | Shampoo | NaCl | | Hand | measure- |
| | | | | | 0.5% WS | stability with | content | | At 0.2% | ment |
| Exam- | | Weight ratio | | | waxy feel | 0.5% WS/ | (% by | Appearance of | WS in | At 0.2% WS |
| ple | Constituents | of monomers | SC % | K value | +++ | 40° C. | wt.) | shampoo | shampoo | in shampoo |
| 001 | VI/VP/Poly- | 22.5:22.5:55 | 26.8 | 72.2 | ++ | stable for at | 1.0% | milky, slightly | 1.2 | 63% |
| | DADMAC | | | | +++ | least 3 | | viscous, | | |
| | | | | | | months | | structure smooth | | |
| 002 | VI/ | 40:60 | 24.1 | 74.2 | ++ | stable for at | 1.0% | milky, slightly | 1.5 | 56% |
| | Poly DADMAC | | | | | least 3 | | viscous, | | |
| | | | | | | months with | | structure smooth | | |
| | | | | | | 1 and 1.5% | | | | |
| | | | | | | by wt. NaCl | | | | |
| 003 | VI/VP/Poly- | 25:25:50 | 26.7 | 72.2 | no | stable for at | 1.0% | milky, viscous, | 1.3 | 54% |
| | DADMAC | | | | | least 3 | | structure smooth | | |
| | | | | | | months | | | | |
| 004 | VI/Poly- | 50:50 | 27.3 | 73 | ++ | stable for at | 1.0% | milky, considerably | 1.5 | 52% |
| | DADMAC | | | | | least 3 | | viscous, structure | | |
| | | | | | | months with | | smooth | | |
| | | | | | | 1 and 1.5% | | | | |
| | | | | | | by wt. of | | | | |
| | | | | | | NaCl | | | | |
| 005 | VI/VP/Poly- | 20:20:60 | 24.8 | 73.7 | ++ | stable for at | 1.0% | milky, viscous, | 1.2 | 63% |
| | DADMAC | | | | | least 3 | | structure smooth | | |
| | | | | | | months | | | | |
| 006 | VI/VP/Poly- | 22.5:22.5:55 | 29.0 | 77 | 0.2% WS | unstable for 3 | 1.0% | milky, slightly | 1.5 | 53% |
| | DADMAC | | | | barely | months | | viscous | | |
| | | | | | waxy | | | structure smooth | | |
| | | | | | 0.5% WS | | | | | |
| | | | | | ++ | | | | | |
| 007 | as 006 but | 22.5:22.5:55 | 36.3 | 78 | ++ | unstable for 3 | 1.0% | milky, slightly | 1.2 | 74% |
| | higher SC | | | | | months and | | viscous, | | |
| | | | | | | the telquel | | structure smooth | | |
| | | | | | | sample also | | | | |
| | | | | | | unstable at | | | | |
| | | | | | | 50° C. | | | | |
| 008 | Poly- | 20/2.5/2.5/7.5 | 29.7 | | no | stable for at | 1.0% | milky, viscous, | 1.5 | 59% |
| | DADMAC/VP/ | | | | | least 3 | | structure smooth | | |
| | Quat 311/Na | | | | | months | | | | |
| | citrate | | | | | | | | | |
| 009 | Poly- | 20/5.0/5.0/7.5 | 37.7 | | no | stable for at | 1.0% | milky, viscous- | 1.5 | 53% |
| | DADMAC/ | | | | | least 3 | | considerably | | |
| | VP/Quat 311/ | | | | | months | | viscous, | | |
| | Na citrate | | | | | | | structure smooth | | |
| 010 | Poly- | 17.5/10/10/6.5 | 43 | | no | stable for at | 1.0% | milky, viscous, | 1.5 | 55% |
| | DADMAC/ | | | | | least 3 | | structure smooth | | |
| | VP/Quat 311/ | | | | | months | | | | |
| | Na citrate | | | | | | | | | |
| 011 | Poly- | 20/5.0/5.0/7.5 | 40.5 | | ++ | stable for at | 1.0% | milky, viscous, | 1.3 | 60% |
| | DADMAC/ | | | | | least 3 | | structure smooth | | |
| | VP/TMAEMC/ | | | | | months | | | | |
| | Na citrate | | | | | | | | | |
| 012 | Poly-DADMAC/ | 20/3.0/7.0/7.5 | 39 | | ++ | stable for at | 1.0% | milky, viscous, | 1.2 | 71% |
| | VP/TMAEMC/ | | | | | least 3 | | structure smooth | | |
| | Na citrate | | | | | months | | | | |
| 013 | Poly- | 20/5.0/5.0/7.5 | 35.8 | | ++ | stable for at | 1.0% | milky, viscous, | 1 | 82% |
| | DADMAC/ | | | | | least 3 | | structure smooth | | |
| | VP/TMAEMC/ | | | | | months | | | | |
| | Na citrate | | | | | | | | | |
| | Batch | | | | | | | | | |
| | procedure | | | | | | | | | |

SC: Solids content
WS: Fraction of polymer combination, calculated as solid

Assessment:

| | | Wet combability | | |
|---|---|---|---|---|
| | | treated by hand | Combing force | |
| Shampoo | Appearance of shampoo | (grade 1-3) | decrease | Feel of the wet hair |
| 0.2% by wt. of example 1 with | Considerably cloudy to milky, | 1- | 66% ± 4 | Very cared for, slightly |
| 1% by wt. of NaCl | viscous, structure smooth | | | waxy |
| 0.5% by wt. of example 2 with | Milky, slightly viscous-viscous, | 1 | 80% ± 3 | Very cared for, waxy |
| 1% by wt. of NaCl | structure smooth | | | |
| 1% by wt. of example 3 with | Milky, white, slightly viscous, | 1 | 83% ± 2 | Very cared for, waxy, |
| 1% by wt. of NaCl | structure smooth | | | somewhat oily |
| 0.2% WS Jaguar C 14 S | Cloudy, viscous, structure | 2 | 25% ± 4 | Cared for |
| with 1% by wt. of NaCl | smooth | | | |
| 0.5% WS Jaguar C 14 S | Cloudy, viscous, structure | 1-2 | 54% ± 4 | Very cared for |
| with 1% by wt. of NaCl | smooth | | | |
| 0.2% WS Jaguar C 14 S + 2% | Milky, slightly viscous-viscous, | 1- | 68% ± 4 | Very cared for, waxy, |
| WS GE Silikon SM 2725 | structure smooth, separated | | | somewhat oily |
| 1% by wt. of NaCl | after 3 weeks since viscosity too | | | |
| | low | | | |
| 0.2% WS Jaguar C 13 S + 2% | Milky, viscous, structure smooth | 1 | 74% ± 4 | Very cared for, waxy, |
| WS GE Silikon SM 2725 | | | | somewhat oily |
| 1.5% by wt. of NaCl | | | | |
| 0.2% WS Jaguar C 14 S + 1% | Milky, viscous, structure smooth | 1 | 68% ± 4 | Very cared for, waxy, |
| WS GE Silikon SM 2725 | | | | somewhat oily |
| 1.5% by wt. of NaCl | | | | |

### Determination of the K Values

The K values were measured in accordance with Fikentscher, Cellulosechemie, Vol. 13, pp. 58 to 64 (1932) at 25° C. in aqueous solution and are a measure of the molecular weight. The solution of the polymers comprises 1 g of polymer in 100 ml of solution.

The K value is measured in a micro-Ubbelohde capillary type M Ic from Schott.

Wet combability (European, bleached hair tresses):

### Determination of Blank Value

Before the determination, the bleached hair tress (length about 24 cm/weight 2.7-3.3 g) was firstly shampooed twice with Texapon®NSO for a total of 1 minute and rinsed for 1 minute in order to achieve a defined wetness and swelling.

Then, the tress was precombed such that knots were no longer present in the hair.

The tress was then fixed to the holder and combed into the fine-toothed side of the test comb using the fine-toothed side of the comb. The hair was placed in the test comb for each measurement evenly and free from tension.

The measurement was started and evaluated using the EGRANUDO® software (Frank). The measurement was repeated 5-10 times. The measurements were carried out in a climatically controlled room at about 65% relative humidity and 21° C.

The calculated average was noted together with the standard deviation.

### Shampoo Formulation:

| | | |
|---|---|---|
| 35.70 | g | Texapon ®NSO |
| 12.50 | g | Tego Betain L 7 |
| 0.5 | g | Polymer combination effective (0.5 g calculated as solid) |
| 0.10 | g | Euxyl ®K 100 |
| ad 100 | g | Water |
| 1.00 | g | NaCl |

5 g of the shampoo to be tested were applied, shampooed for 1 min, rinsed for 1 min, squeezed on filter paper and combed, and the measurement was determined.

### Evaluation:

Combing force decrease wet=100−(measurement*100/blank value); data in %

Instruments used: Stress/strain tester from Frank

Digital balance (top-pan balance)

### Examples of Cosmetic Compositions:

### Hair Cosmetic Composition (General)

a) 0.01 to 5% by weight of the polymer combination (calculated as solid)

b) 25 to 99.99% by weight of water and/or alcohol

c) 0 to 95.99% by weight of further constituents

Alcohol is understood as meaning all alcohols customary in cosmetics, e.g. ethanol, isopropanol, n-propanol.

Further constituents are to be understood as meaning the additives customary in cosmetics, for example propellants, antifoams, interface-active compounds, i.e. surfactants, emulsifiers, foam formers and solubilizers. The interface-active compounds used may be anionic, cationic, amphoteric or neutral. Further customary constituents may also be, for example, preservatives, perfume oils, opacifiers, active ingredients, UV filters, care substances, such as panthenol, collagen, vitamins, protein hydrolyzates, alpha- and beta-hydroxycarboxylic acids, chitosan, protein hydrolyzates, cosmetic polymers, stabilizers, pH regulators, dyes, viscosity regulators, gel formers, dyes, salts, humectants, refatting agents, complexing agents and further customary additives.

### Shampoo Formulation/Shower Gel Formulation

Preferred shampoo formulations or shower gel formulations comprise

a) 0.01 to 5% by weight of the polymer combination (calculated as solid)

b) 25 to 99.99% by weight of water

c) 0-5% by weight of a further conditioning agent

d) 0-30% by weight of further cosmetic constituents

Furthermore, all anionic, neutral, amphoteric or cationic surfactants used customarily in shampoos can be used in the shampoo formulations with the above provisos.

### Example 1

### Conditioner Shampoo with PQ-10

| | | |
|---|---|---|
| 35.70 | g | Sodium laureth sulfate |
| 6.50 | g | Cocamidopropylbetaine |
| 0.20 | g | Polymer combination according to example 1 |
| 0.40 | g | Polyquaternium-10 |
| 0.10 | g | Preservative |
| 0.10 | g | Perfume oil/essential oil |
| ad 100 | g | Aqua dem. |

Good conditioner shampoos are also obtained if, instead of the polymer combination according to example 1, the polymer combinations of examples 2 to 13 are used.

### Example 2

### Conditioner Shampoo with GHTC

| | | |
|---|---|---|
| 35.70 | g | Sodium laureth sulfate |
| 6.50 | g | Cocamidopropylbetaine |
| 0.50 | g | Polymer combination according to example 1 |
| 0.20 | g | Guar hydroxypropyltrimonium chloride |
| 0.10 | g | Preservative |
| 0.10 | g | Perfume oil/essential oil |
| ad 100 | g | Aqua dem. |

Good conditioner shampoos are also obtained if, instead of the polymer combination according to example 1, the polymer combinations of examples 2 to 13 are used.

### Example 3

### Conditioner Shampoo with Polyquaternium

| | | |
|---|---|---|
| 35.70 | g | Sodium laureth sulfate |
| 6.50 | g | Cocamidopropylbetaine |
| 0.20 | g | Polymer combination according to example 1 |
| 0.30 | g | Polyquaternium-44 or PQ-67 |
| 0.10 | g | Preservative |
| 0.10 | g | Perfume oil/essential oil |
| ad 100 | g | Aqua dem. |

Good conditioner shampoos are also obtained if, instead of the polymer combination according to example 1, the polymer combinations of examples 2 to 13 are used.

### Example 4

### Shampoo

Phase A

| 15.00 | g | Cocamidopropylbetaine |
|---|---|---|
| 10.00 | g | Disodium cocoamphodiacetate |
| 5.00 | g | Polysorbate 20 |
| 5.00 | g | Decyl glucoside |
| 0.20 | g | Polymer combination according to example 1 |
| 0.10 | g | Perfume oil/essential oil |
| q.s. | | Preservative |
| 2.00 | g | Laureth-3 |

| | |
|---|---|
| ad 100 | Aqua dem. |
| q.s. | Citric acid |

### Phase B

3.00 g PEG-150 distearate

### Preparation

Weigh in components of phase A and dissolve; adjust pH to 6-7. Add phase B and heat to 50° C. Allow to cool to room temperature with stirring.

Good shampoos are also obtained if, instead of the polymer combination according to example 1, the polymer combinations of examples 2 to 13 are used.

### Example 5

### Shampoo

| | |
|---|---|
| 30.00 g | Sodium laureth sulfate |
| 6.00 g | Sodium cocoamphoacetate |
| 0.50 g | Polymer combination according to example 1 |
| 3.00 g | Sodium laureth sulfate, glycol distearate, cocamide MEA, |
| | laureth-10 |
| 2.00 g | Dimethicone |
| q.s. | Perfume |
| q.s. | Preservative |
| q.s. | Citric acid |
| 1.00 g | Sodium chloride |
| ad 100 | Aqua dem. |

Good shampoos are also obtained if, instead of the polymer combination according to example 1, the polymer combinations of examples 2 to 13 are used.

### Example 6

### Shower Gel

| | |
|---|---|
| 20.00 g | Ammonium laureth sulfate |
| 15.00 g | Ammonium lauryl sulfate |
| 0.50 g | Polymer combination according to example 1 |
| 0.50 g | Polyquaternium-7 |
| 2.50 g | Sodium laureth sulfate, glycol distearate, cocamide MEA, |
| | laureth-10 |
| 0.10 g | Perfume oil/essential oil |
| q.s. | Preservative |
| 0.50 g | Sodium chloride |
| ad 100 | Aqua dem. |

Good shower gels are also obtained if, instead of the polymer combination according to example 1, the polymer combinations of examples 2 to 13 are used.

### Example 7

### Shower Gel

| | |
|---|---|
| 40.00 g | Sodium laureth sulfate |
| 5.00 g | Decyl glucoside |
| 5.00 g | Polymer combination according to example 1 |
| 1.00 g | Panthenol |
| 0.10 g | Perfume oil/essential oil |
| q.s. | Preservative |
| q.s. | Citric acid |
| 2.00 g | Sodium chloride |
| ad 100 | Aqua dem. |

Good shower gels are also obtained if, instead of the polymer combination according to example 1, the polymer combinations of examples 2 to 13 are used.

### Example 8

### Shampoo

| | |
|---|---|
| 12.00 g | Sodium laureth sulfate |
| 1.50 g | Decyl glucoside |
| 0.50 g | Polymer combination according to example 1 |
| 5.00 g | Cocoglucoside glyceryl oleate |
| 2.00 g | Sodium laureth sulfate, glycol distearate, cocomide MEA, |
| | laureth-10 |
| q.s. | Preservative |
| q.s. | Sunset Yellow C. I. 15 985 |
| 0.10 g | Perfume oil/essential oil |
| 1.00 g | Sodium chloride |
| ad 100 | Aqua dem. |

Good shampoos are also obtained if, instead of the polymer combination according to example 1, the polymer combinations of examples 2 to 13 are used.

The polymer combinations for the use according to the invention are also suitable for use in hairstyling preparations, in particular hair foams (aerosol foams with propellent gas and pump foams without propellent gas), hairsprays (pump sprays without propellent gas) and hair gels.

Propellants are the customarily used propellants. Preference is given to mixtures of propane/butane, pentane, dimethyl ether, 1,1-difluoroethane (HFC-152 a), carbon dioxide, nitrogen or compressed air.

### Aerosol Hair Foam

a) 0.1 to 10% by weight of a cosmetic polymer

b) 55 to 99.8% by weight of water and/or alcohol

c) 5 to 20% by weight of a propellant

d) 0.1 to 5% by weight of a polymer combination suitable according to the invention

e) 0 to 10% by weight of further constituents

Further constituents which may be used are, inter alia, all emulsifiers used customarily in hair foams. Suitable emulsifiers may be nonionic, cationic or anionic or amphoteric.

Examples of nonionic emulsifiers (INCI nomenclature) are laureths, e.g. laureth-4; ceteths, e.g. ceteth-1, polyethylene glycol cetyl ether; ceteareths, e.g. ceteareth-25, polyglycol fatty acid glycerides, hydroxylated lecithin, lactyl esters of fatty acids, alkyl polyglycosides.

Examples of cationic emulsifiers are cetyldimethyl-2-hydroxyethylammonium dihydrogenphosphate, cetyltrimonium chloride, cetyltrimonium bromide, cocotrimonium methyl sulfate, quaternium-1 to x (INCI).

Anionic emulsifiers can be selected, for example, from the group of alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

A preparation suitable according to the invention for styling gels can, for example, have the composition as follows:

### Example 9

### Aerosol Hair Foam

| | |
|---|---|
| 2.00 g | Cocotrimonium methosulfate |
| 0.10 g | Perfume oil/essential oil |
| 3.50 g | Setting polymer, e.g. polyquaternium-46, PQ-44, |
| | VP/methacrylamide/vinylimidazole copolymer, etc. |
| 0.80 g | Polymer combination according to example 1 |
| q.s. | Preservative |
| 75.00 g | Water dem. |
| 10.00 g | Propane/butane (3.5 bar) |

Good aerosol hair foams are also obtained if, instead of the polymer combination according to example 1, the polymer combinations of examples 2 to 13 are used.

### Styling Gel

a) 0.1 to 10% by weight of a cosmetic polymer

b) 60 to 99.85% by weight of water and/or alcohol

c) 0.05 to 10% by weight of a gel former

d) 0.1 to 5% by weight of a polymer combination suitable according to the invention

e) 0 to 20% by weight of further constituents

Gel formers which can be used are all gel formers customary in cosmetics. These include slightly crosslinked polyacrylic acid, for example carbomer (INCI), cellulose derivatives, e.g. hydroxypropylcellulose, hydroxyethylcellulose, cationically modified celluloses, polysaccharides, e.g. xanthan gum, caprylic/capric triglycerides, sodium acrylates copolymer, polyquaternium-32 (and) paraffinum liquidum (INCI), sodium acrylates copolymer (and) paraffinum liquidum (and) PPG-1 trideceth-6, acrylamidopropyltrimonium chloride/acrylamide copolymer, steareth-10 allyl ether acrylates copolymer, polyquaternium-37 (and) paraffinum liquidum (and) PPG-1 trideceth-6, polyquaternium-37 (and) propylene glycol dicaprate dicaprylate (and) PPG-1 trideceth-6, polyquaternium-7, polyquaternium-44, polyquaternium-67.

Good styling gels are also obtained if, instead of the polymer combination according to example 1, the polymer combinations of examples 2 to 13 are used.

### Example 10

### Hairstyling Gel

Phase A

| | |
|---|---|
| 0.50 g | Carbomer or acrylates/C10-30 alkyl acrylate |
| | crosspolymer |
| 86.40 g | Water dem. |

Phase B

| | |
|---|---|
| 0.70 g | Triethanolamine |

Phase C

| | |
|---|---|
| 6.00 g | Setting polymer, e.g. VP/methacrylamide/ |
| | vinylimidazole copolymer |
| 5.00 g | PVP |
| 0.20 g | PEG-25 PABA |
| 0.50 g | Polymer combination according to example 1 |
| 0.10 g | Perfume oil/essential oil |
| q.s. | PEG-14 dimethicone |
| q.s. | Preservative |
| 0.10 g | Tocopheryl acetate |

Good styling gels are also obtained if, instead of the polymer combination according to example 1, the polymer combinations of examples 2 to 13 are used.

### Example 11

### Hairstyling Gel

Phase A

| | |
|---|---|
| 0.50 g | Carbomer or acrylates/C10-30 alkyl acrylate crosspolymer |
| 91.20 g | Water dem. |

Phase B

| | |
|---|---|
| 0.90 g | Tetrahydroxypropylethylenediamine |

Phase C

| | |
|---|---|
| 7.00 g | VP/VA copolymer |
| 0.40 g | Polymer combination according to example 1 |
| 0.20 g | Perfume oil/essential oil |
| q.s. | Preservative |
| 0.10 g | Propylene glycol |

Good styling gels are also obtained if, instead of the polymer combination according to example 1, the polymer combinations of examples 2 to 13 are used.

### Example 12

### Hair Wax Cream

| 6.00 | g | Caprylic/capric triglycerides |
|---|---|---|
| 3.00 | g | Glyceryl stearate |
| 2.00 | g | Cetyl alcohol |
| 3.50 | g | Polymer combination according to example 1 |
| 0.50 | g | Cremophor A6 |
| 0.70 | g | Cremophor A25 |
| 0.50 | g | Dimethicone |
| 0.50 | g | Vitamin E acetate |
| 2.00 | g | Caprylic/capric triglycerides and sodium acrylates |
| | | copolymer |
| 1.00 | g | D-panthenol USP |
| 0.10 | g | EDTA |
| 10.00 | g | Setting polymer |
| q.s. | | Preservative |
| ad 100 | g | Water dem. |

Good hair wax creams are also obtained if, instead of the polymer combination according to example 1, the polymer combinations of examples 2 to 13 are used.

### Example 13

### Hair Pudding

| 3.00 | g | Kollicoat IR (BASF) |
|---|---|---|
| q.s. | | Preservative |
| 2.00 | g | Setting polymer |
| 4.00 | g | Acrylates/beheneth-25 methacrylate copolymer |
| 0.70 | g | Polymer combination according to example 1 |
| 0.50 | g | Dimethicone copolyol |
| 0.10 | g | EDTA |
| 0.20 | g | Benzophenone-4 |
| ad 100 | g | Water dem. |

Good hair puddings are also obtained if, instead of the polymer combination according to example 1, the polymer combinations of examples 2 to 13 are used.

### Example 14

### Spray Gel

Phase A

| | |
|---|---|
| 1.25 g | Setting polymer |
| 96.15 g | Aqua dem. |

Phase B

| | |
|---|---|
| 0.70 g | Acrylates/steareth-20 itaconate copolymer |
| 0.10 g | Propylene glycol |
| 0.50 g | Polymer combination according to example 1 |
| 0.10 g | Glycerol |
| 0.10 g | Perfume oil/essential oil |
| q.s. | Preservative |

Phase C

| | |
|---|---|
| 0.70 g | Triethanolamine |

Good spray gels are also obtained if, instead of the polymer combination according to example 1, the polymer combinations of examples 2 to 13 are used.

A preparation suitable according to the invention for styling sprays can, for example, have the composition as follows:

### Example 15

### Pump Hair Spray

| | | |
|---|---|---|
| 11.20 | g | PEG/PPG-25/25 dimethicone/acrylates copolymer |
| 2.80 | g | VP/VA copolymer |
| 1.34 | g | Aminomethylpropanol |
| 0.30 | g | Polymer combination according to example 1 |
| 0.10 | g | Perfume oil/essential oil |
| 11.26 | g | Aqua dem. |
| 73.00 | g | Alcohol |

Good pump hair sprays are also obtained if, instead of the polymer combination according to example 1, the polymer combinations of examples 2 to 13 are used.

### Example 16

### Pump hair spray VOC55

| | |
|---|---|
| 2.00 g | VP/methacrylamide/vinylimidazole copolymer |
| 1.90 g | Polyquaternium-46 |
| 2.00 g | Polymer combination according to example 1 |
| 0.10 g | Perfume oil/essential oil |
| 55.00 g | Alcohol |
| 39.00 g | Aqua dem. |

Good pump hair sprays VOC 55 are also obtained if, instead of the polymer combination according to example 1, the polymer combinations of examples 2 to 13 are used.

### Skin Cosmetic Compositions

### Example 17

### Liquid Makeup

Phase A

| | |
|---|---|
| 1.70 g | Glyceryl stearate |
| 1.70 g | Cetyl alcohol |
| 1.70 g | Ceteareth-6 |
| 1.70 g | Ceteareth-25 |
| 5.20 g | Caprylic/capric triglycerides |
| 5.20 g | Mineral oil or Luvitol ® Lite (INCI Hydrogenated |
| | Polyisobutene) |

Phase B

| | |
|---|---|
| q.s. | Preservative |
| 4.30 g | Propylene glycol |
| 2.50 g | Polymer combination according to example 1 |
| 59.50 g | Aqua dem. |

Phase C

| | |
|---|---|
| 0.10 g | Perfume oil/essential oil |

Phase D

| | |
|---|---|
| 2.00 g | Iron oxides |
| 12.00 g | Titanium dioxide |

Good liquid makeups are also obtained if, instead of the polymer combination according to example 1, the polymer combinations of examples 2 to 13 are used.

### Example 18

### Eyeliner

Phase A

| | | |
|---|---|---|
| 40.60 | g | dist. water |
| 0.20 | g | Disodium EDTA |

| | |
|---|---|
| q.s. | Preservative |

Phase B

| | | |
|---|---|---|
| 0.60 | g | Xanthan Gum |
| 0.40 | g | Veegum |
| 3.00 | g | Butylene glycol |
| 0.20 | g | Polysorbate-20 |

Phase C

| | | |
|---|---|---|
| 15.00 | g | Iron oxide/Al powder/silica (e.g. Sicopearl ® |
| | | Fantastico Gold from BASF) |

Phase D

| | | |
|---|---|---|
| 10.00 | g | Aqua dem. |
| 25.00 | g | Setting polymer (e.g. polyurethane-1 or VP/ |
| | | methacrylamide/vinylimidazole copolymer, etc.) |
| 5.00 | g | Polymer combination according to example 1 |

Good eyeliners are also obtained if, instead of the polymer combination according to example 1, the polymer combinations of examples 2 to 13 are used.

### Example 19

### Sunscreen Gel

Phase A

| | |
|---|---|
| 0.90 g | Polymer combination according to example 1 |
| 8.00 g | Octyl methoxycinnamate |
| 5.00 g | Octocrylene |
| 0.80 g | Octyltriazone |
| 2.00 g | Butylmethoxydibenzoylmethane |
| 2.00 g | Tocopheryl acetate |
| 0.10 g | Perfume oil/essential oil |

Phase B

| | |
|---|---|
| 0.30 g | Acrylates/C10-30 alkyl acrylate crosspolymer |
| 0.20 g | Carbomer |
| 5.00 g | Glycerol |
| 0.20 g | Disodium EDTA |
| q.s. | Preservative |
| 75.30 g | Aqua dem. |

Phase C

| | |
|---|---|
| 0.20 g | Sodium hydroxide |

Good sunscreen gels are also obtained if, instead of the polymer combination according to example 1, the polymer combinations of examples 2 to 13 are used.

### Example 20

### Sunscreen emulsion with TiO₂ and ZnO₂

Phase A

| | |
|---|---|
| 1.00 g | PEG-7 hydrogenated castor oil |
| 5.00 g | Polymer combination according to example 1 |
| 2.00 g | PEG-45/dodecyl glycol copolymer |
| 3.00 g | Isopropyl myristate |
| 7.90 g | Jojoba (Buxus Chinensis) oil |
| 4.00 g | Octyl methoxycinnamate |
| 2.00 g | 4-Methylbenzylidenecamphor |
| 3.00 g | Titanium dioxide, dimethicone |
| 1.00 g | Dimethicone |
| 5.00 g | Zinc oxide, dimethicone |

Phase B

| | |
|---|---|
| 0.20 g | Disodium EDTA |
| 5.00 g | Glycerol |
| q.s. | Preservative |
| 60.80 g | Aqua dem. |

Phase C

| | |
|---|---|
| 0.10 g | Perfume oil/essential oil |

Good sunscreen emulsions are also obtained if, instead of the polymer combination according to example 1, the polymer combinations of examples 2 to 13 are used.

### Example 21

### Face Tonics

Phase A

| | |
|---|---|
| 3.00 g | Polymer combination according to example 1 |
| 0.10 g | Perfume oil/essential oil |
| 0.30 g | Bisabolol |

Phase B

| | |
|---|---|
| 3.00 g | Glycerol |
| 1.00 g | Hydroxyethylcetyldimonium phosphate |
| 5.00 g | Witch Hazel (Hamamelis Virginiana) distillate |
| 0.50 g | Panthenol |
| q.s. | Preservative |
| 87.60 g | Aqua dem. |

Good face tonics are also obtained if, instead of the polymer combination according to example 1, the polymer combinations of examples 2 to 13 are used.

### Example 22

### Face Washing Paste with Peeling Effect

Phase A

| | |
|---|---|
| 73.00 g | Aqua dem. |
| 1.50 g | Carbomer |
| q.s. | Preservative |

Phase B

| | |
|---|---|
| q.s. | Perfume oil |
| 7.00 g | Potassium cocoyl hydrolyzed protein |
| 4.00 g | Polymer combination according to example 1 |

Phase C

| | |
|---|---|
| 1.50 g | Triethanolamine |

Phase D

| | |
|---|---|
| 13.00 g | Polyethylene (Luwax A ™ from BASF) |

Good face washing pastes are also obtained if, instead of the polymer combination according to example 1, the polymer combinations of examples 2 to 13 are used.

### Example 23

### Soap

Phase A

| | | |
|---|---|---|
| 25.00 | g | Potassium cocoate |
| 20.00 | g | Disodium cocoamphodiacetate |
| 2.00 | g | Lauramide DEA |
| 1.0 | g | Glycol stearate |
| 2.00 | g | Polymer combination according to example 1 |
| 50.00 | g | Aqua dem. |

| | |
|---|---|
| q.s. | Citric acid |

Phase B

| q.s. | | Preservative |
|---|---|---|
| 0.10 | g | Perfume oil/essential oil |

Good soaps are also obtained if, instead of the polymer combination according to example 1, the polymer combinations of examples 2 to 13 are used.

### Example 24

### Face Cleansing Milk O/W Type

Phase A

| | | |
|---|---|---|
| 1.50 | g | Ceteareth-6 |
| 1.50 | g | Ceteareth-25 |
| 2.00 | g | Glyceryl stearate |
| 2.00 | g | Cetyl alcohol |
| 10.00 | g | Mineral oil |

Phase B

| 5.00 | g | Propylene glycol |
|---|---|---|
| q.s. | | Preservative |
| 1.00 | g | Polymer combination according to example 1 |
| 66.30 | g | Aqua dem. |

Phase C

| | | |
|---|---|---|
| 0.20 | g | Carbomer |
| 10.00 | g | Cetearyl octanoate |

Phase D

| | | |
|---|---|---|
| 0.40 | g | Tetrahydroxypropylethylenediamine |

Phase E

| | | |
|---|---|---|
| 0.10 | g | Perfume oil/essential oil |
| 0.10 | g | Bisabolol |

Good face cleansing milks are also obtained if, instead of the polymer combination according to example 1, the polymer combinations of examples 2 to 13 are used.

### Example 25

### Transparent Soap

| | | |
|---|---|---|
| 4.20 | g | Sodium hydroxide |
| 3.60 | g | dist. water |
| 10.00 | g | Polymer combination according to example 1 |
| 22.60 | g | Propylene glycol |
| 18.70 | g | Glycerol |
| 5.20 | g | Cocoamide DEA |
| 2.40 | g | Cocamine oxide |
| 4.20 | g | Sodium lauryl sulfate |
| 7.30 | g | Myristic acid |
| 16.60 | g | Stearic acid |
| 5.20 | g | Tocopherol |

Good transparent soaps are also obtained if, instead of the polymer combination according to example 1, the polymer combinations of examples 2 to 13 are used.

### Example 26

### Shaving Foam

| | |
|---|---|
| 6.00 g | Ceteareth-25 |
| 5.00 g | Poloxamer 407 |
| 52.00 g | Aqua dem. |
| 1.00 g | Triethanolamine |
| 5.00 g | Propylene glycol |
| 1.00 g | PEG-75 lanolin oil |
| 5.00 g | Polymer combination according to example 1 |
| q.s. | Preservative |
| 0.10 g | Perfume oil/essential oil |
| 25.00 g | Sodium laureth sulfate |

Bottling: 90 parts of active substance and 10 parts of a 25:75 propane/butane mixture.

Good shaving foams are also obtained if, instead of the polymer combination according to example 1, the polymer combinations of examples 2 to 13 are used.

### Example 27

### Aftershave Balm

Phase A

| 0.25 | g | Acrylates/C10-30 alkyl acrylate crosspolymer |
|---|---|---|
| 1.50 | g | Tocopheryl acetate |
| 0.20 | g | Bisabolol |
| 10.00 | g | Caprylic/capric triglycerides |
| q.s. | | Perfume |
| 1.00 | g | Polymer combination according to example 1 |

Phase B

| | | |
|---|---|---|
| 1.00 | g | Panthenol |
| 15.00 | g | Alcohol |
| 5.00 | g | Glycerol |
| 0.05 | g | Hydroxyethylcellulose |
| 1.90 | g | Polymer combination according to example 1 |
| 64.02 | g | dist. water |

Phase C

| | | |
|---|---|---|
| 0.08 | g | Sodium hydroxide |

Good aftershave balms are also obtained if, instead of the polymer combination according to example 1, the polymer combinations of examples 2 to 13 are used.

### Example 28

### Care Cream

Phase A

| | |
|---|---|
| 2.00 g | Ceteareth-6 |
| 2.00 g | Ceteareth-25 |
| 2.00 g | Cetearyl alcohol |
| 3.00 g | Glyceryl stearate SE |
| 5.00 g | Mineral oil |
| 4.00 g | Jojoba (Buxus Chinensis) oil |
| 3.00 g | Cetearyl octanoate |
| 1.00 g | Dimethicone |
| 3.00 g | Mineral oil, lanolin alcohol |

Phase B

| | |
|---|---|
| 5.00 g | Propylene glycol |
| 0.50 g | Veegum |
| 1.00 g | Panthenol |
| 1.70 g | Polymer combination according to example 1 |
| 6.00 g | Polyquaternium-44 |
| q.s. | Preservative |
| 60.80 g | Aqua dem. |

Phase C

| | |
|---|---|
| q.s. | Perfume |

Good care creams are also obtained if, instead of the polymer combination according to example 1, the polymer combinations of examples 2 to 13 are used.

### Oral and Dental Care Preparations

### Example 29

### Toothpaste

Phase A

| | | |
|---|---|---|
| 34.79 | g | Aqua dem. |
| 3.00 | g | Polymer combination according to example 1 |
| 20.00 | g | Glycerol |
| 0.76 | g | Sodium monofluorophosphate |

Phase B

| | | |
|---|---|---|
| 1.20 | g | Sodium carboxymethylcellulose |

Phase C

| 0.80 | g | Aroma oil |
|---|---|---|
| 0.06 | g | Saccharin |
| q.s. | | Preservative |
| 0.05 | g | Bisabolol |
| 1.00 | g | Panthenol |
| 0.50 | g | Tocopheryl acetate |
| 2.80 | g | Silica |
| 1.00 | g | Sodium lauryl sulfate |
| 7.90 | g | Dicalcium phosphate anhydrate |
| 25.29 | g | Dicalcium phosphate dihydrate |
| 0.45 | g | Titanium dioxide |

Good toothpastes are also obtained if, instead of the polymer combination according to example 1, the polymer combinations of examples 2 to 13 are used.

### Example 30

### Mouthwash

Phase A

| | |
|---|---|
| 2.00 g | Aroma oil |
| 4.50 g | Polymer combination according to example 1 |
| 1.00 g | Bisabolol |
| 30.00 g | Alcohol |

Phase B

| | |
|---|---|
| 0.20 g | Saccharin |
| 5.00 g | Glycerol |
| q.s. | Preservative |
| 5.00 g | Poloxamer 407 |
| 52.30 g | Aqua dem. |

Good mouthwashes are also obtained if, instead of the polymer combination according to example 1, the polymer combinations of examples 2 to 13 are used.

### Example 37

### Prosthesis Adhesive

Phase A

| 0.20 | g | Bisabolol |
|---|---|---|
| 1.00 | g | Beta-carotene |
| q.s. | | Aroma oil |
| 20.00 | g | Cetearyl octanoate |
| 5.00 | g | Silica |
| 33.80 | g | Mineral oil |

Phase B

| | | |
|---|---|---|
| 5.00 | g | Polymer combination according to example 1 |
| 35.00 | g | PVP (20% strength solution in water) |

Good prosthesis adhesives are also obtained if, instead of the polymer combination according to example 1, the polymer combinations of examples 2 to 13 are used.

The examples below are prepared, for example, as described in U.S. Pat. No. 6,451,300, columns 31 and 32.

| Example 50 | % by wt. |
|---|---|
| Ammonium laureth sulfate | 12 |
| Ammonium lauryl sulfate | 8 |
| Polymer combination according to example 1 | 0.4 |
| PEG-90M³ (INCI) | 0.5 |
| Zinc pyrithione⁴ (optional) | 1 |
| 1-Decene homopolymers⁵ | 0.3 |
| Trimethylpropan etricaprylate/caprate⁶ | 0.1 |
| Dimethicones⁷ (optional) | 2.0 |
| Ethylene glycol distearate | 2.0 |
| Cocoamide MEA | 0.8 |
| Cetyl alcohol | 0.9 |
| Water and lesser amounts | q.s. |

³PEG M_{w} ca. 4*10⁶ g/mol
⁴Zinc pyrithione with average particle size of ca. 2.5 μm;
⁵Puresyn 6 (hydrogenated polydecene)
⁶Mobil ® P43
⁷Visasil ® 330 000 cSt (General Electric Silicones).

Good shampoos are also obtained if, instead of the polymer combination according to example 1, the polymer combinations of examples 2 to 13 are used.

| | Example | |
|---|---|---|
| | 63 | 64 |
| | % by wt. | % by wt. |
| Ammonium laureth sulfate | 12 | 10 |
| Ammonium lauryl sulfate | 6 | 6 |
| Polyquaternium-10 | — | 0.25 |
| Polymer combination according to example 1 | 0.25 | 0.25 |
| PEG-7M⁴ (INCI) | — | 0.1 |
| PEG-90M⁵ (INCI) | 0.1 | — |
| Zinc pyrithione⁶ (optional) | 1 | 1 |
| 1-Decene homopolymers⁷ | 0.4 | 0.4 |
| Trimethylpropan etricaprylate/caprate⁸ | — | 0.1 |
| Dimethicones⁹ (optional) | 1.15 | 1.35 |
| Ethylene glycol distearate | 1.0 | 1.5 |
| Cocoamide MEA | 1.1 | 0.8 |
| Cetyl alcohol | 0.6 | 0.9 |
| Water and lesser amounts | q.s. | q.s. |

⁴Polyox ® WSR N-750
⁵Polyox ® WSR N-301
⁶Zinc pyrithione with average particle size of ca. 2.5 μm;
⁷Puresyn 6 (hydrogenated polydecene)
⁸Mobil ® P43
⁹Visasil ® 330 000 cSt (General Electric Silicones).

Good shampoos are also obtained in each case if, instead of the polymer combination according to example 1, the polymer combinations of examples 2 to 13 are used.

| Example 90 | % by wt. |
|---|---|
| Sodium laureth sulfate | 10 |
| Sodium lauryl sulfate | 6 |
| Ethylene glycol distearate | 1.5 |
| Cocoamide MEA | 0.8 |
| Cetyl alcohol | 0.9 |
| Polymer combination according to example 1 | 0.5 |
| Dimethicones⁹ (optional) | 2.35 |
| Trimethylpropan etricaprylate/caprate⁸ | 0.1 |
| 1-Decene homopolymers⁷ | 0.4 |
| Zinc pyrithione¹⁰ (optional) | 1.0 |
| Sodium citrate | 0.2 |
| Citric acid | 0.22 |
| Sodium chloride | 1.475 |
| Perfume | 0.7 |
| Sodium benzoate | 0.25 |
| Kathon ® CG | 0.0005 |
| Water | q.s. |

¹⁰Zinc pyrithione with average particle size of ca. 2.5 μm;
⁷Puresyn 6 (hydrogenated polydecene)
⁸Mobil ® P43
⁹Visasil ® 330 000 cSt (General Electric Silicones).

Good shampoos are also obtained if, instead of the polymer combination according to example 1, the polymer combinations of examples 2 to 13 are used.

### Example 103

### Hair Repair Shampoo “Smooth & Silky”

| | % by | | | |
|---|---|---|---|---|
| Phase | wt. | Ingredient | Source | INCI name |
| A | 0.5 | Polymer combination | | |
| | | according to example 1 | | |
| | 47.3 | Water demineralized | | Aqua |
| B | 12.5 | Tego ® Betain L7 | Evonik | Cocamidopropyl |
| | | | Goldschmidt | Betaine |
| | | | GmbH | |
| | 35.7 | Texapon ® NSO | Cognis | Sodium Laureth |
| | | | Deutschland | Sulfate |
| | | | GmbH | |
| | | | & Co. KG | |
| | 0.10 | Euxyl ® K 100 | Schülke | Benzyl |
| | | | & Mayr | Alcohol (and) |
| | | | GmbH | Methyl |
| | | | | chloroiso- |
| | | | | thiazolinone |
| | | | | (and) Methyl |
| | | | | chloroiso- |
| | | | | thiazolinone |
| | 0.30 | Perfume | | Fragrance |
| | 0.50 | D-Panthenol USP | BASF SE | Panthenol |
| | 0.10 | Edeta ® BD | BASF SE | Disodium EDTA |
| | 1.5 | NaCl | | Sodium Chloride |

Preparation: add the components of phase B one after the other to the mixed phase A and stir until all of the components have completely dissolved.

Properties: pH 6.5, viscosity: 9000 mPa·s (Brookfield DV II+sp. 4/20 rpm)

Good shampoos are also obtained if, instead of the polymer combination according to example 1, the polymer combinations of examples 2 to 13 are used.

1.-18. (canceled)

19. A composition comprising
a) at least one polymer a) with cationic and/or cationogenic groups and a molecular weight M_{w} in the range from 10 000 to 5 million and
b) at least one polymer b) selected from
b1) polymers prepared in the presence of polymer a) which comprise a compound of the formula I in copolymerized form 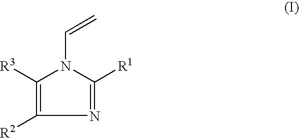
where R¹ to R³, independently of one another, are hydrogen, C₁-C₄-alkyl or phenyl, and
b2) cationic polymers prepared in the presence of polymer a) and at least one salt and comprising, in copolymerized form, at least one cationic monomer and at least one compound of the formula I.

20. The composition according to claim 19, wherein polymer a) comprises diallyldimethylammonium chloride in copolymerized form.

21. The composition according to claim 19, wherein polymer a) consists of at least 50% by weight of copolymerized diallyldimethylammonium chloride.

22. The composition according to claim 19, wherein polymer a) consists of at least 70% by weight of copolymerized diallyldimethylammonium chloride and polymer a) has a molecular weight M_{w} in the range from 100 000 to 200 000 g/mol.

23. The composition according to claim 19, wherein polymer a) has a molecular weight M_{w}, in the range from 50 000 to 500 000 g/mol.

24. The composition according to claim 19, wherein polymer b1) comprises, in copolymerized form,
i) in the range from 10 to 100% by weight, at least one compound of the formula I and
ii) in the range from 0 to 90% by weight, at least one compound of the formula II 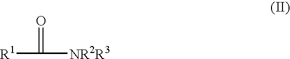
where R¹ is a group of the formula CH₂═CR⁴- where R⁴═H or C₁-C₄-alkyl, and R² and R³, independently of one another, are H, alkyl, cycloalkyl, heterocycloalkyl, aryl or hetaryl, or R² and R³, together with the nitrogen atom to which they are bonded, are a five- to eight-membered nitrogen heterocycle or
R² is a group of the formula CH₂═CR⁴—, and R¹ and R³, independently of one another, are H, alkyl, cycloalkyl, heterocycloalkyl, aryl or heteraryl, or R¹ and R³, together with the amide group to which they are bonded, are a lactam having 5 to 8 ring atoms.

25. The composition according to claim 19, wherein the ratio of the weight amounts of the polymers a) and b) in the composition is in the range from 4:1 to 1:4.

26. The composition according to claim 19, wherein the ratio of the weight amounts of the polymers a) and b) in the composition is in the range from 3:2 to 2:3.

27. The composition according to claim 19, wherein polymer b) consists of at least 10% by weight of copolymerized N-vinylimidazole.

28. The composition according to claim 19, wherein polymer b) consists of at least 40% by weight of copolymerized N-vinylpyrrolidone.

29. The composition according to claim 19, wherein polymer b1) has a molecular weight M_{w} in the range from 1500 to 500 000 g/mol.

30. The composition according to claim 19, wherein the cationic polymer b2) comprises at least one quaternized compound of the formula III in copolymerized form 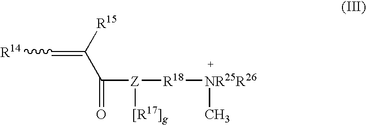
where
R¹⁴ and R¹⁵, independently of one another, are selected from the group consisting of hydrogen, C₁-C₈ linear- or branched-chain alkyl, methoxy, ethoxy, 2-hydroxyethoxy, 2-methoxyethoxy and 2-ethoxyethyl,
R¹⁷ is hydrogen or methyl,
R¹⁸ is alkylene or hydroxyalkylene having 1 to 24 carbon atoms, optionally substituted by alkyl,
g is 0 or 1,
Z is nitrogen when g=1 or oxygen when g=0,
R²⁵ and R²⁶, in each case and independently of one another, are selected from the group consisting of hydrogen, C₁-C₄₀ linear- or branched-chain alkyl, formyl, C₁-C₁₀ linear- or branched-chain acyl, N,N-dimethylaminoethyl, 2-hydroxyethyl, 2-methoxyethyl, 2-ethoxyethyl, hydroxypropyl, methoxypropyl, ethoxypropyl or benzyl.

31. The composition according to claim 30, wherein R¹⁸ is C₂H₄, C₃H₆, C₄H₈, CH₂—CH(OH)—CH₂.

32. The composition according to claim 28, where the quaternized compound is selected from the group consisting of methylated N-[3-(dimethylamino)propyl]methacrylamide and methylated N,N-dimethylaminoethyl methacrylate.

33. The composition according to claim 19, where the total amount of polymer a) and polymer b) in the composition is in the range from 0.01 to 20% by weight, based on the weight of the composition.

34. The composition according to claim 19, further comprising at least one anionic surfactant.

35. A haircare composition comprising the composition according to claim 19.

36. A skin cosmetic composition comprising the composition according to claim 19.

37. The haircare composition according to claim 35 in the form of a shampoo.

38. A method of producing the composition according to claim 19, wherein polymer b1) is produced by solution polymerization in the presence of polymer a).
